(19) United States
(12) Patent Application Publication
(10) Pub. No.: US 2002/0137141 A1
(43) Pub. Date: Sep. 26, 2002

(54) Short peptides from the 'A-region' of protein kinases which selectively modulate protein kinase activity

(75) Inventor: Shmuel Ben-Sasson, Jerusalem (IL)

Correspondence Address: BROWDY AND NEIMARK, P.L.L.C., 624 NINTH STREET, NW, SUITE 300, WASHINGTON, DC, 20001-5303, US

(73) Assignee: Children's Medical Center Corporation, Boston, MA (US)

(21) Appl. No.: 10/012,034

(22) Filed: Dec. 11, 2001

Related U.S. Application Data
(63) Continuation-in-part of application No. 09/734,520, filed on Dec. 11, 2000.

(51) Int. Cl. C12Q 1/68; C12N 9/12; A61K 38/16; C12P 21/02

(52) U.S. Cl. 435/69.1; 514/12; 435/6; 435/194

(57) ABSTRACT

The present invention concerns compounds comprising, within short sequences from a specific region of the kinase, that can modulate kinase-associated signal transduction.

### CROSS-REFERENCE TO RELATED APPLICATIONS

This application is a continuation-in-part of U.S. application Ser. No. 09/734,520, filed Dec. 11, 2000, the content of which is hereby incorporated entirely by reference.

### FIELD OF THE INVENTION

The present invention concerns compounds for modulating kinase associated signal transduction. The invention further concerns methods for using said compounds as well as methods for identifying and synthesizing said compounds.

### BACKGROUND OF THE INVENTION

The eukaryotic protein kinase superfamily is composed of enzymes which specifically phophorylate serine, threonine or tyrosine residues of intracellular proteins. These enzymes are important in mediating signal transduction in multicellular organisms and are involved in a wide variety of cellular events. A few examples include: cellular proliferation, cellular differentiation, oncogenesis, immune responses, and inflammatory responses.

Enhanced protein kinase activity can lead to persistent stimulation by secreted growth factors and other growth inducing factors which, in turn, can lead to proliferative diseases such as cancer, to nonmalignant proliferative diseases such as arteriosclerosis, psoriasis and to inflammatory responses such as septic shock. Decreased function can also lead to various diseases.

Thus, agents that can modulate (increase or decrease) the activity of protein kinases have great potential for the treatment of a wide variety of diseases and conditions such as cancer, autoimmune disorders, and inflammation.

PKs are known to have homologous “catalytic domains” which are responsible to the phosphorylation activity. Based on a comparison of a large number of protein kinases, it is now known that the kinase domain of protein kinases can be divided into twelve subdomains. These are regions that are generally uninterrupted by large amino acid insertions and contain characteristic patterns of conserved residues (Hanks and Hunter, “The Eukaryotic Protein Kinase Superfamily”, in Hardie and Hanks ed., The Protein Kinase Facts Book, Volume I, Academic Press, Chapter 2, 1995). These subdomains are referred to as Subdomain I through Subdomain XII.

Due to the high degree of homology found in the subdomains of different protein kinases, the amino acid sequences of the domains of different PKs can be aligned. Frequently, the alignment is carried out with reference to the prototypical protein kinase PKA-Cα, as known in the art. Currently, the catalytic domains of a large number of protein kinases have been aligned and tables showing these alignments are available from various published sources, such as, for example, in the article by Hanks and Quinn in Methods of Enzymology 200: 38-62 (1991) or in the PKR Web Site: WWW.sdsc.edu/kinases.

### SUMMARY OF THE INVENTION

The present invention is based on the discovery that compounds comprising relatively short sequences, identical to native sequences appearing in a specific region of a kinase (hereinafter “A-region of a kinase”), or variants of said native sequences, are capable of altering the signal transduction mediated by the same kinase from which the sequences were obtained. Thus the invention leads to the discovery of compound that can modulate a signal transduction associated with a kinase in a specific manner unique to said kinase.

Without wishing to be bound by theory, it is assumed that the compounds of the invention are active through one of several mechanisms. According to one mechanism the compound binds to the kinase and by this increases or decreases directly the activity of the kinase. Decrease of the activity may be for example due to masking a domain required for interaction with other proteins, or by conferring an unfavorable conformational change in the kinase leading to decrease in the activity of the enzyme. Increase of the kinase activity, may be due, for example, to the induction of a conformational change in the kinase that renders it more active. Where the kinase activity is mediated by dimerization of two or more kinases, the increase of activity may be due to the fact that the compound of the invention mimics one kinase when binding to the other, so that its presence when bound to a kinase is sufficient to simulate dimerization.

An alternative mechanism of action is based on the preferred assumption, in accordance with the invention, that the peptidic portion of the compound of the invention mimics a region (A-region) in the kinase that interacts with other cellular components, such as the substrates of the kinase, or with phosphatases or other kinases (of the same or of a different type) that de-phosphorylate, or phosphorylate, respectively, the specific kinase. This mimic sequence, when present in the compound of the invention, is then assumed to bind to the other cellular component (not to the kinase) and by this interrupts the interaction of the native kinase with the cellular components. Where originally the interaction between the kinase and the cellular component is an “on” interaction (for example phosphorylation of a substrate resulting in increased transcription) said interruption causes inhibition of the signal transduction mediated by the kinase. Where the interaction between the kinase the cellular component is a “off” reaction (such as the interaction of a kinase with a phosphatase which dephosphorylates and decreases the activity of the kinase) said interruption of interaction decreases the “off” direction, resulting in an increase in signal transduction mediated by the kinase (for example increased phosphorylation, increased activity in glucose uptake, etc.).

It has further been found in accordance with the invention that for the purpose of modulating the activity of the kinase it is possible to prepare a compound comprising any one of several short subsequences appearing in the A-region, or variants of the sequences having some alterations as compared to the native sequence. In accordance with the above assumption of the invention, the activity of the mimic sequence is to interrupt the interaction between the kinase and other cellular components. For such an interruption there is no need to faithfully copy the full region and mimicking of one of several optional sub-parts of the regions is sufficient. Furthermore it probably is sufficient merely to copy the overall structure of the region, as well as the chemical properties of those amino acids in the regions responsible for the protein-protein interaction, to obtain modulating properties. This explains the fact that many times a variant having many alterations as compared to the native sequence has the same, or even better modulating properties than the native sequence. The improvement in activity of the variant may be due for example to stabilization of a more favorable conformations

Thus, the present invention allows for the first time a method for readily identifying compounds that are candidates for modulating signal transduction associated with a kinase.

The present invention also enables obtaining compounds that can modulate said kinase-mediated signal transduction, by testing the candidates, and selecting from the candidates only those compounds which modulated said kinase-associated signal transduction.

The present invention also concerns a method for the modulation of kinase-associated signal transduction comprising the administration of said compounds. This method may be used for the treatment of a plurality of diseases that are caused, by or are a result of non-normal kinase activity.

The present invention also concern compounds for the modulation of kinase-associated signal transduction, as well as pharmaceutical compositions comprising these compounds.

The present invention also concerns the use of said compounds for the reparation of medicaments.

### GENERAL DESCRIPTION OF THE INVENTION

By one aspect, the present invention concerns a method for identifying candidate compounds for the modulation of signal transduction associated with a kinase, the method comprising:

(a) identifying a peptide region in the kinase (“A-region”) by aligning catalytic subunits of the kinase and PKA-Cα and determining the sequence of the kinase corresponding to positions 92-109 of PKA-Cα;

(b) synthesizing at least one compound comprising a sequence selected from:

(b1) a sequence comprising of from a minimum of 5 continuous amino acids of said A-region to a maximum of all the continuous amino acids of said A-region;

(b2) a variant of the sequence of (b1) wherein up to 40% of the amino acids of the sequence of (b1) have been replaced with a naturally or non-naturally occurring amino acid or with a peptidomimetic organic moiety; and/or up to 40% of the amino acids have their side chains chemically modified, and/or up to 20% of the amino acids have been deleted, provided that at least 50% of the amino acids of (b1) are maintained unaltered in the variant;

(b3) a sequence of (b1) or (b2) wherein one or more of the amino acids is replaced by the corresponding D-amino acid;

(b4) a sequence of any one of (b1) to (b3) wherein at least one peptidic backbone atom, or peptidic backbone bond has been altered to a modified peptidic backbone atom or a non-naturally occurring peptidic backbone bond, respectively;

(b5) a sequence of any one of (b1), (b2), (b3) or (b4) in a reverse order; and

(b6) a combination of two or more of the sequences of (b1), (b2), (b3), (b4) or (b5).

(c ) testing each compound of (b) to determine the capacity thereof to modulate the signal transduction associated with the kinase.

Preferably the determination of the signal transduction associated with the kinase is by determination of the level of phosphorylation of at least one kinase-substrate, and step (c) comprises subjecting cellular components of the signal transduction to the presence or absence of the compound, and determinating whether the presence of said compound caused change in the level of phosphorylation of the least one substrate as compared to the level of phosphorylation in the absence of the compound.

The present invention also concerns a method for obtaining a compound for the modulation of kinase-associated signal transduction the method comprising:

(a) identifying candidate compounds for the modulation of kinase associated kinase transduction as defined above;

(b) selecting from the candidate compounds those compounds which modulate signal transduction associated in the test assay, as compared to the modulation in the same test assay in the absence of the candidate compound; and

(c) producing the selected compounds of (b) thereby obtaining compounds for the modulation of kinase associated signal transduction.

The present invention also concerns compounds for the modulation of kinase associated signal transduction obtained by the above method.

The present invention further concerns a method for modulating signal transduction associated with a kinase by administrating a compound obtained by any of the above methods.

The present invention still further concerns a method for the treatment of a disease, disorder or condition, wherein a therapeutically beneficial effect may be evident by the modulation of at least one signal transduction associated with a kinase comprising: administering to a subject in need of such treatment a therapeutically effective amount of the above compound.

The term “signal transduction associated with the kinase” refers to the level of signaling of a specific signaling pathway wherein the specific kinase is one of the effectors of the signaling. The determination of the signal transduction is carried out by determination of the phosphorylation level. Said level of signaling may be determined directly by measuring the level of phosphorylation of a substrate for kinase phosphorylation, in a response to a given signal. The substrate may be the direct substrate of the kinase to be modulated, or may be another substrate in the signaling pathway, that is more downstream than the direct substrate of the kinase (sometimes it is more convenient to check phosphorylation of a more downstream kinase). The measurement may be also carried out by measuring other indirect biochemical, cellular or physiological properties which are changed as a result of the signal transduction associated with the kinase as will be explained in the Detailed Description part of the specification.

This modulation may be caused by a direct effects on the kinase itself (for example due to binding to the kinase) or alternatively and preferably, as explained above, the modulation may be caused by the interruption of the interaction of the kinase with various cellular components (such as substrates, cofactors, regulators, other kinases and other phosphatases), by the binding of the compound to the cellular components, and said interruption may lead to the modulation in the signal transduction.

The term “modulating” (modulation to modulate etc.) refers to an increase, or decrease, in the level of the signal transduction associated with the kinase, as determined by any of the assays. For example, if the signal transduction is determined by assessing the level of phosphorylation of a specific substrate (which may be either the direct substrate of the kinase in question, or a substrate of another kinase more downstream in the pathway) modulation refers to increase, or decrease in the level of phosphorylation as compared to the level of phosphorylation in the same assay in the absence of the compound of the invention (or in the presence of a control compound).

The term :“cellular components of the signal transduction” refers to the molecules that participate in the signal transduction in which the kinase is involved including: the receptor, the kinase, other kinases, phosphatases, substrates (which may be also the same or other kinases) co-factors, ATP and effector molecules.

The term “compound” (comprising sequence)” refers to a compound that includes within any of the sequences of (b1) to (b6) as defined above. The compound may be composed mainly from amino acid residues, and in that case the amino acid component of the compounds should comprise no more than a total of about 35 amino acids. Where the compound is mainly an amino acid molecule, it may consist of any one of the amino acid sequences of (b1) to (b5) a combination of at least two, preferably three, most preferably of two, of the sequences of (b1) to (b5) linked to each other (either directly or via a spacer moiety) to give (b6). The compound may further comprise any one of the amino acids sequences, or combinations as described above (in (b1) to (b6) above), together with additional amino acids or amino acid sequences other than those of (b1) to (b6). The additional amino acids may be sequences from other regions of the kinase, sequences that are present in the kinase in vicinity of the A-region, N-terminal or C-terminal to the sequences defined in (a), or sequences which are not present in the specific kinase but were included in the compound in order to improve various physiological properties such as: penetration into cells (sequences which enhance penetration through membranes); decreased degradation or clearance; decreased repulsion by various cellular pumps, improved immunogenic activities, improvement in various modes of administration (such as attachment of various sequences which allow penetration through various barriers such as the blood-brain barrier, through the gut, etc.); increased specificity, increased affinity, decreased toxicity, moieties added for imaging purposes and the like. A specific example is the addition of the amino acid Gly or several Gly-residues in tandem to N-terminal of the sequence.

The compound may also comprise non-amino acid moieties, such as for example, hydrophobic moieties (various linear, branched cyclic, polycyclic or hetrocyclic hydrocarbons and hydrocarbon derivatives) associated to the peptides of (b1) to (b6) to improve penetration through membranes; various protecting groups, especially where the compound is a linear molecule, attached to the compound's terminals to decreased degradation; chemical groups present in the compound to improve penetration or decrease toxic side effects, or various spacers, placed for example, between one or more of the above amino acid sequences, so as to spatially position them in suitable order in respect of each other and the like. The compound of the invention may be a linear or cyclic molecule, and cyclization may take place by any means known in the art. Where the compound is composed predominantly of amino acids/amino acid sequences, cyclization may N- to C-terminal, N-terminal to side chain and N-terminal to backbone, C-terminal to side chain, C-terminal to backbone, side chain to backbone and side chain to side chain, as well as backbone to backbone cyclization. Cyclization of the molecule may also take place through the non-amino acid organic moieties.

The association between the A-region-derived sequence (defined in (b1) to (b6) and other components of the compound may be by covalent linking, or by non-covalent complexion, for example, by complexion to a hydrophilic polymer, which can be degraded or cleaved thereby producing a compound capable of sustained release (the cleavage may be inside the cell thus releasing the peptidic portion of the compound), by entrapping the peptidic part of the compound in liposomes or micelles to produce the final compound of the invention, etc.

The term “a sequence comprising of from 5 continuous amino acids of said A-region to a maximum of . . . ” means any continuous stretch of at least 5 amino acids, which are present in a longer amino acid sequence described by reference to positions of PKA-Cα (see below). For example, if in a specific kinase, the positions corresponding to amino acid residues 92-109 are amino acid residues 200 to 217 of that specific kinase, the continuous stretch of at least 5 amino acids may be from amino acid at position 200 to 204, from 201 to 205, from 213 to 217, etc. The continuous sequence may be of 5, 6, (for example 200-205 . . . , 212-117), 7 (200-206 . . . 217), 8, 9, 10, 11, 12, 13, 14, 15, 16, 17 or 18 amino acids. Preferably the sequences are 7 to 13 continuous amino acids of the A-region.

The term “sequence corresponding to positions . . . to . . . of PKA-C_{α}” refers to a sequence that is matches the sequence appearing in the native PKA-Cα when the catalytic units of the two are aligned. For determining the beginning and end positions of the specific kinase used, the sequence of the catalytic unit of the specific kinase should be aligned with the sequence of the catalytic unit of PKA-Cα in pair-wise or multiple-alignment manner. Alignment may be carried out using any state of the art software such as ClustAl™ (version W or X). Alternatively for producing the alignment it is possible to use tables showing these alignments which are available from various published sources, such as, for example, in the article by Hanks and Quinn in Methods of Enzymology 200: 38-62 (1991) or in the PKR Web Site: WWW.sdsc.edu/kinases. In some kinases extra amino or less amino acids may be present in this region and the size of the A-region can, therefore, include more or less than 18 amino acids in length, however, the alignment methods (both present in ready table or carried out by known programs) can be carried out even if the size of the A-region of the kinase and the A-region of PKA-Cα are different. It shall be noted that when the kinase is PKA-Cα itself the positions are already given.

A complementary manner for identifying the A-region, which can help in case the alignment is problematic is by reference to the three-dimensional structure of the kinase. It is possible to identify in the kinase the beginning of the B4-beta sheet, identifying the amino acid of the kinase in that position, and determining the A-region as a sequence about 18 amino acids N-terminal to the beginning of the B4 sheet. In terms of the three dimensional structure of kinases, the kinase domain of PKs has been found to contain at least nine alpha helices, referred to as helix A through helix I and nine beta sheets, referred to as (b1) through (b9) (Tabor et al., Phil Trans. R. Soc. Lond. B340:315 (1993), Mohammadi et al, Cell, 86:577 (1996) and Hubbard et al., Nature, 372:746 (1994)). Relationships between the primary structure of a large number of protein kinases and their corresponding three dimensional structure is well known in the

The term “a variant wherein up to 40% of the amino acids of the native sequence have been replaced with a naturally or non-naturally occurring amino acid or with a peptidomimetic organic moiety” in accordance with the present invention, concerns a peptide, which corresponds in at least about 60% of its amino acid with the native sequence as described in (b1) above, but some (up to 40%) of the amino acids were replaced either by other naturally occurring amino acids, (both conservative and non-conservative substitutions), by non-naturally occurring amino acids (both conservative and non-conservative substitutions), or with organic moieties which serve either as true peptidomimetics (i.e. having the same steric and electrochemical properties as the replaced amino acid), or merely serve as spacers in lieu of a deleted amino acid, so as to keep the spatial relations between the amino acid spanning this replaced amino acid. Generally, essential amino acids as determined by various Structure-Activity-Relationship (SAR) techniques (for example amino acids hast when replaced by Ala cause loss of activity) are replaced by conservative substitution while non essential amino acids can be deleted or replaced by any type of substitution. Guidelines for the determination of the deletions, replacements and substitutions are given in the detailed description part of the specification. Preferably no more than 35%, 30, 25% or 20% have been replaced.

The term “wherein up to 40% of the amino acids have their side chains chemically modified” refers to a variant which has the same type of amino acid residue as in the native sequence, but to its side chain a functional groups has been added. For example, the side chain may be phosphorylated, glycosylated, fatty acylated, acylated, iodinated or carboxyacylated. Other examples of chemical substitutions are known in the art and some are given below.Preferably no more than 35%, 30%, 25%, or 20% of the amino acids have their side chains chemically modified.

The term “up to 20% of the amino acids have been deleted” refer to an amino acid sequence which maintains at least 20% of its amino acid. Preferably no more than 10% of the amino acids are deleted and more preferably none of the amino acids are deleted.

The term “provided that at least 50% of the amino acids in the parent protein are maintained unaltered in the variants ” the up to 40% substitution, up to 40% chemical modification and up to 20% deletions are combinatorial, i.e. the same variant may have substitutions, chemical modifications and deletions so long as at least 50% of the amino acids of the variant are identical (in nature and in position) to those of the native sequence. In addition, the properties of the parent sequence, in modulating kinase-associated signal transduction, have to be maintained in the variant typically at the same or higher level.

When calculating 40% (or 35, 30, 25, 20%) replacement of 20% (or 10%) deletion from sequences, the number of actual amino acids should be rounded mathematically, so that both 40% of an 11 mer sequence (4.4) and 40% of a 12 mer sequence (4.8) is four amino acids, and only 40% of a 13 mer sequence (5.2) is five amino acids.

The term “at least one peptidic backbone atom or peptidic backbone bone have been chemically modified or altered to a non-naturally occurring peptidic backbone bond, respectively” means that the bond between the N- of one amino acid residue to the C- of the next has been altered to non-naturally occurring bonds by reduction (to —CH₂—NH—), alkylation (methylation) on the nitrogen atom, or the bonds have been replaced by amidic bond, urea bonds, or sulfonamide bond, etheric bond (—CH₂—O—), thioetheric bond (—CH₂—S—), or to —CS—NH—,; The side chain of the residue may be shifted to the backbone nitrogen to obtain N-alkylated-Gly (a peptidoid). Preferably all the peptidic backbone has been altered to make the compound more resistant to degradation.

The term “where one or more of the amino acids is replaced by the corresponding D-amino acid” refers to replacement of a specific amino acid X in a normal L-configuration by the D-counterpart. In particular for producing “retro inverso” (see below).

The term “in reverse order” refers to the fact that the sequence of (b1), (b2) or (b3) or (b4) may have the order of the amino acids as it appears in the native kinase from N- C direction, or may have the reversed order (as read in the C- to N-direction) for example, if a continuous stretch of 5 amino acids from the A-loop of TGFβ receptor 1 is QTVML a sequence in a reverse order is LMVTQ. It has been found that many times sequences having such a reverse order can have the same properties in short peptides as the “correct” order, probably due to the fact that the side chains, and not the peptidic backbone are those responsible for the interactions. Particularly preferred, are what is termed “retro inverso” peptides—i.e. peptides that have both a reverse order as explained above, and in addition each and every single one of the amino acids, has been replaced by the non-naturally occurring D- amino acid counterpart, so that the net end result as regards the positioning of the side chains (the combination of reverse order and as the change from L to D) is zero change. Such retro-inverso peptides, while having similar binding properties to the native peptide, were found to be resistant to degradation.

The combination may be of two or more, more preferably three, most preferably two of the sequences of (b1) to (b5).

Examples of kinase which signal transduction can be modulated by the method of the invention include, but are not limited to, PKs belonging to the following kinase families: Src associated kinases, endothelial growth factor receptors, fibroblast growth factor receptors (FGFRs), hepatic growth factor receptors (HGFRs), epidermal growth factor receptors (EGFRs), neural growth factor receptors (NGFRs), Janus kinases (JAKs), Activin receptor-like kinases (ALKs), discoidin domain receptors (DDRs), Ephrin receptors (EphRs), insulin and IGF receptor kinases and Polo family kinases. Suitable members from the Src kinase family include, but are not limited to, Src, Yes, Fyn, Fgr, Lyn, Hck, Lck, Csk and Matk. Suitable members from the endothelial growth factor receptors family include, but are not limited to Tie, Tek, PDGF receptor a and b, Flt 1 and 4 and Flk1. Suitable members from the FGFR family include, but are not limited to, Flg, Bek, FGFR3 and FGFR4. Suitable members from the ALK family include, but are not limited to, ALK1, ALK2, ALK3, ALK4, ALK5, and ALK6. Suitable members from the HGFR family include, but are not limited to, c-Met, c-Sea and Ron. Suitable members from the EGFR family include, but are not limited to, EGFR, ErbB2, ErbB3 and ErbB4. Suitable members from the NGFR family include, but are not limited to, Trk-NGFR, TrkB and TrkC. Suitable members from the JAK family include, but are not limited to, Jak1, Jak2, Jak3 and Tyk2. Suitable members from the DDR family include, but are not limited to, DDR1 and DDR2. Suitable members from the EphR family include, but are not limited to, Eph-B4. Suitable members from the Polo family include, but are not limited to, Plk, Plx1, polo, SNK, CDC5, Sak, Prk, Fnk and Plo1. Other suitable PKs include, but are not limited to, focal adhesion kinase (FAK), c-Ab1, Ret, insulin receptor kinase (IRK), Syk and Zap70, ACK and TEC.

As shown in FIG. 1, the sequences of an A-region of kinases from different families include, but are not limited to: Src, Yes, Fyn, Fgr, Lyn, Hck, Lck (SEQ ID NO. 1 to 7); Csk and Matk (SEQ ID NO. 8 to 9); focal adhesion kinase (FAK) (SEQ ID NO. 10); c-Ab1 (SEQ ID NO. 11); endothelial growth factor receptors Tie, Tek, FGF receptor (Flg, Bek, FGFR3, FGFR4), PDGF receptors a and b, Flt 1 and 4 and Flk1 (SEQ ID NO. 12 to 19); HGF receptors c-Met, c-Sea and Ron (SEQ ID NO. 20 to 22); EGF receptor (EGFR, ErbB2, ErbB3, ErbB4) (SEQ ID NO. 23 to 26); Ret (SEQ ID NO. 27); NGF receptors (Trk) (SEQ ID NO. 28 to 29); Syk and Zap70 (SEQ ID NO. 30 to 31); Jak kinases 1 through 3 and Tyk2 (SEQ ID NO. 32 to 35); insulin receptor kinase (IRK) (SEQ ID NO. 36 and 123-133); Activin receptor-like kinases 1 through 6 (ALK1, 2, 3, 4, 5, 6) (SEQ ID NO. 37 to 40); discoidin domain receptors 1 and 2 (DDR) (SEQ ID NO. 41 to 42); ACK (SEQ ID NO. 43); Ephrin receptor B4 (SEQ ID NO. 44); TEC (SEQ ID NO. 45); Polo family kinases Plk, Plx1, polo, SNK, CDC5, Sak, Prk, Fnk and Plo1 (SEQ ID NO. 46 to 53).

The amino acid at the N-terminus of the A region is at position 1 and can be referred to as “[AA]₁”. The next amino acid in the sequence, referred to as “[AA]₂”, is at position 2 and is followed by amino acids [AA]₃ through [AA]ₘ, which are at positions 3 to m, where m is the position number of the amino acid at the C-terminus of the A-region. Likewise, (m-12) is the position number of the amino acid twelve amino acid residues before the C-terminus of the A-region. Thus, a peptide 18-aa with an amino acid sequence [AA₁] through [AA₁₈] includes the first eighteen amino acids in the A-region. A peptide derivative of the A-region with an amino acid sequence [AA₅] through [AA₁₆] includes the fifth amino acid through the sixteenth amino acid in the A-region, and a peptide derivative of the A-region with an amino acid sequence [AA]₍ₘ₋₁₂₎ through [AA]ₘ includes the last twelve amino acids in the A-region. m can have a value between 5 and 18. This terminology will be used in the claims.

The present invention includes molecules comprising sequences that have been varied as explained above. The native sequences of the kinases that can be varied are selected from: Plk; Plx1; polo; SNK; CDC5; Sak; Prk; Plo1; ALK1; ALK2; ALK3; c-Src; c-Yes; Fyn; c-Fgr; Lyn; Hck; Lck; Csk; Matk; Fak; c-Ab1; Tie; PDGFR-b; PDGFR-a; Flt1; Flt4; Flg; FGFR-4; c-Met; c-Sea; Ron; EGFR; ErbB2; ErbB3; ErbB4; Ret; Trk-NGFR; TrkB; Syk; Zap70; Jak1; Jak2; Jak3; IRK; DDR1; DDR2; Tyk2; Eph-B4; ITK/TSK and ACK.

Specific examples of sequences to be included in the compounds of the present invention include: Plk K035A100; Plx1 K036A100; polo K037A100; SNK K038A100; CDC5 K039A100; Sak K040A100; Prk K041A100; Plo1 K043A100; ALK1 K048A100; c-Src K051A100; c-Yes K052A100; Fyn K053A100; c-Fgr K054A100; Lyn K055A100; Hck K056A100; Lck K057A100; Csk K058A100; Matk K059A100; Fak K060A100; c-Ab1 K061A100; Tie K062A100; PDGFR-b K064A100; PDGFR-a K065A100; Flt1 K066A100; Flt4 K067A100; Flg K069A100; FGFR-4 K072A100; c-Met K073A100; c-Sea K074A100; Ron K075A100; EGFR K076A100; ErbB2 K077A100; ErbB3 K078A100; ErbB4 K079A100; Ret K080A100; Trk-NGFR K081A101 K081A102 K081A103 K081A104; Syk K082A100; Zap70 K083A100; Jak1 K084A100; Jak2 K085A100; Jak3 K086A100; IRK K094A103 K094A104 K094A105 K094A106 K094A107 K094A108 K094A112 K094A113 K094A114 K094A115 K094A116 K094A117 K094A118; K094A119; K094A131; K094A132; K094A122; ALK2 K097A100; ALK3 K098A100; TrkB K102A100; DDR1 K104A100; DDR2 K105A100; Tyk2 K108A100; Eph-B4 K114A100; ITK/TSK K140A100; ACK K141A100 (SEQ ID NO. 54 to 122, respectively), as specified in FIG. 3, or SEQ ID NO: 123.

The N-terminus and/or C-terminus of these sequences present in the compounds can be modified, as described above and as shown in FIG. 3. The N-terminal of these peptides can be myristylated and the C-terminal is amidated. Other protecting groups for amides and carboxylic acids can be used, as will be described bellow. Optionally, one or both protecting groups can be omitted. The compounds may be linear or cyclic.

The signal transduction associated with the kinase in a subject can be modulated for treating a disease condition or disorder, wherein a beneficial effect may be evident by the modulation of kinase activity. For example, the treatment may be of diseases that are caused by over-activity or under-activity of kinases. For example, inhibition of c-Met or tyrosine kinase receptors which respond to fibroblast growth factor (FGF) or vascular endothelial growth factor (VEGF) decreases angiogenesis. In addition, RET is involved in certain thyroid cancers. Compounds of the present invention which modulate the activity of these enzymes can be used to treat cancer in a subject when administered to the subject in a therapeutically effective amount.

Restenosis is caused by vascular smooth muscle proliferation in response to, for example, vascular injury caused by balloon catheterization. Vascular smooth muscle proliferation is also a cause of atherosclerosis. Vascular smooth muscle proliferation is a result of, for example, inhibition of CSK and/or stimulation of tyrosine kinase receptors which respond to FGF or platelet derived growth factor (PDGF). Thus, restenosis and atherosclerosis can be treated with a therapeutically effective amounts of a compound which inhibits tyrosine kinase receptors which respond to FGF or PDGF or which activate CSK.

FGF has also been implicated in psoriasis, arthritis and benign prostatic hypertrophy (Dionne et al., WO 92/00999). These conditions can be treated with a molecule of the invention.

Src activity is responsible, at least in part, for bone resorption. Thus, osteoporosis can be treated with a therapeutically effective amount of a peptide or peptide derivative which inhibits Src activity or which activates Csk.

Lyn and HCK are activated during the non-specific immune response which lo occurs in individuals with arthritis, as a result of autoimmune responses. Lyn is also activated in individuals with septic shock. Thus, these conditions can be treated with a therapeutically effective amount a compound which inhibits the activity of these kinases.

Lck is expressed in T cells and is activated during a T cell immune response. Similarly, Lyn is expressed in B cells and activated during a B-cell immune response. Thus, conditions which are caused by overactivation of T cells or B cells can be treated by administering a therapeutically effective amount of a compound which inhibits Lck or Lyn, respectively. Conditions which are caused by under-activation of T cells or B cells can be treated by administering a therapeutically effective amount of a compound which stimulates Lck or Lyn, respectively. In addition, a severe reduction of the B cell progenitor kinase leads to human X-linked agammaglobulinemia, which can be treated by administering a therapeutically effective amount of a compound which stimulates B cell progenitor kinase.

Decreased function of other kinases can also lead to disease. For example, a decrease in the activity of insulin receptor tyrosine kinase (IRK) is a cause of various types of diabetes and may be involved both in Type I and Type II diabetes. These types of diabetes can be treated by administering a therapeutically effective amount of a compound which increases the activity of the IRK.

Another example of beneficial therapeutical outcome may be in conditions where the activity of the kinase is normal, but nevertheless change of the signal transduction may improve the condition, for example, increase in normal healing rate of bone, skin or connective tissue, leads to improved healing (such as healing without scarring), etc. For example a family of transmembrane protein kinases is composed of members of the TGFβ/Activin/BMP receptors which transduce signals of the corresponding cytokines. The TGFβ/Activin/BMP cytokines participate in various processes of tissue remodelling, including the induction of bone formation, hair growth, adipose tissue proliferation, neural cell stimulation and differentiation of pancreatic islet cells. Therefore, modulation of the activity of these receptor kinases can assist tissue repair, inhibit tissue fibrosis and fat tissue growth, assist in hair growth, induce differentiation of pancreatic β-cells, help neural cell survival and function and enhance bone formation (even in cases where these activities are normal) thus resulting in a therapeutically beneficial effect.

Based on methods disclosed herein, compounds can be designed in the future to modulate the activity of kinase whose A-region has been sequenced or will be sequenced in the future and whose cellular function is known. As a consequence, compounds can be designed to affect (increase or decrease) those cellular functions. It is possible that future research will reveal that certain disease conditions, whose underlying causes are presently unknown, are brought about by the overactivity or underactivity of cellular functions controlled by these kinases. These diseases can be treated by administering compounds comprising sequences obtained from the A-region or variants of the over- or under- active kinase. Compounds can be identified by methods disclosed above.

A “therapeutically effective amount” is the quantity of the compound which results in a “therapeutically beneficial effect” as a result of the treatment compared with a typical clinical outcome in the absence of the treatment.

A “therapeutically beneficial effect” results in the individual with the disease experiencing fewer symptoms or complications of the disease, including a longer life expectancy, as a result of the treatment. With respect to cancer, an “therapeutically beneficial effect” includes a longer life expectancy. It can also include slowing or arresting the rate of growth of a tumor, causing a shrinkage in the size of the tumor, a decreased rate of metastasis and/or improved quality of life (e.g., a decrease in physical discomfort or an increase in mobility).

With respect to diabetes, therapeutically beneficial effect refers to lowering of blood sugar that can result in a longer life expectancy, a reduction in the complications of the disease (e.g., neuropathy, retinopathy, nephropathy and degeneration of blood vessels) and an improved quality of life, as described above. Another aspect of a therapeutically beneficial effect is a reduction in medication dosage (e.g., a reduction in insulin or other hypoglycemic agent needed to maintain adequate blood glucose levels), reduction in the frequency of insulin administration episodes required etc.

With respect to obesity, s therapeutically beneficial effect refers to increased weight reduction per caloric intake or a reduction in food intake. It also refers to a decrease in the complications which are a consequence of obesity, for example heart disease such as arteriosclerosis and high blood pressure.

The method for therapeutic treatment, by way of tissue remodeling may be a condition or disorder selected from bone formation, reduced scar formation, enhanced hair growth, induction of differentiation of pancreatic duct cells, inhibition of the growth of adipose tissue, cancer treatment, diseases caused by proliferation of smooth muscle (e.g. restenosis and atherosclerosis), skin disorders, diabetes, obesity, diseases of the central nervous system, inflammatory disorders, autoimmune diseases and other immune disorders, osteoporosis and cardiovascular diseases.

The term “treatment” in the context of the invention includes: cure of the disease or condition, prevention of the disease before it occurs, or prevention of deterioration of the disease, as well as decrease in the severity of at least one undesired manifestation of the disease.

The amount of compounds of the invention administered to the individual will depend on the type and severity of the disease and on the characteristics of the individual, such as general health, age, sex, body weight and tolerance to drugs as well as on the mode of administration. In the case of tissue remodeling, many applications are local to the tissue, the amounts used when locally administered may be smaller than in systemic administration. The skilled artisan will be able to determine appropriate dosages depending on these and other factors. Typically, a therapeutically effective amount of the compound can range from about 1 mg per day to about 1000 mg per day for an adult. Preferably, the dosage ranges from about 1 mg per day to about 100 mg per day.

It should be appreciated that for the purpose of modulation, one should choose a compound comprising sequences derived from the same member of the kinase known (for example, in literature or from clinical information) to be involved in the specific disease, disorder or condition to be treated, or sequences derived from members of the same kinase family.

It should be appreciated that some of the compounds comprising the sequences of (b1) to (b6) above, are not active in modulating signal transduction associated by the kinase, and the selection of the compounds which are active in the above modulation should be done according to the method as indicated above.

Preferably, the determination of the sequence to be included in the candidate compound for modulating kinase-associated signal transduction should be carried out with the following steps:

(a) determining which specific kinase-associated signal transduction is to be modulated and determining the sequence of the specific kinase from a database of amino acid sequences;

(b) determining the A-region of the kinase by aligning the sequence of the catalytic unit of the kinase determined in (i) with the sequence of the catalytic unit of PKA-Cα, and determining the sequence of the specific kinase corresponding to position 92-107 of PKA-Cα (A-region);

(c) determining a continuous stretch of at least 5 amino acids of any of the A-regions above that is shorter than the length of the full region and modulated the kinase-associated signal transduction, by synthesizing a plurality of subsequences (optionally partially overlapping subsequences) of 5-10 aa obtained from the A-region; testing those subsequences in a test assay for determining signal transduction associated with the kinase, and selecting those subsequences that modulated said signal transduction associated with the kinase;

(d) determining in the sequences of (b) or in the sequences selected in (c) above, essential and non-essential amino acids by: preparing a plurality of modified sequences wherein in each sequence a single and different amino acid of the native sequence has been replaced with a test amino acid (preferably with Ala) to produce modified sequences; testing those modified sequences in a test assay for determining signal transduction associated with the kinase; those amino acids which when replaced, caused a statistically significant change in signal transduction associated with the kinase being non-essential amino acids;

(e) preparing a plurality of compounds comprising sequences selected from:

(1) the sequences of(b);

(2) the sequences selected in (c);

(3) the sequences of (ii) or the selected sequence of (c), wherein at least one of the essential amino acids has been replaced by a conservatively substituted naturally or non-naturally occurring amino acid, or a conservative peptidomimetic organic moiety and/or the sequences of (b) or the selected sequence obtained in (c), wherein at least one of the non-essential amino acids has been deleted, or substituted (conservatively or non-conservatively) by naturally or non-naturally occurring amino acids or a peptidomimetic, or the sequences of (b) or (c) where at least one of the amino acids have been chemically modified;

(4) the sequences of (1) to (3) in a reverse order;

(5) the sequence of 4 wherein all the amino acids have been replaced by their D-counterpart residues;

(6) sequences wherein at least one of the peptidic backbones has been altered to a non-naturally occurring peptidic backbone;

said compounds of (v) being candidate compounds for modulating kinase-associated signal transduction.

Conceptually, the first step is deciding which specific kinase is involved in the kinase-associated signal transduction which is to be modulated, for example by carrying out a literature search, and determining which kinase is known to be involved in the relevant pathway. The sequence of that kinase is the one used to determine the A-region sequence. Many times there is a cascade of several kinases involved in a specific pathway and it is important to decide which specific kinases in the signal transduction pathway should be modulated to give the best effect when deciding such factors, such as: is there a “by-path” signal transduction by other kinases? Will the modulation of the kinase be specific to one pathway, or non specific effects on several pathways?

Once this specific kinase is chosen, its sequence can be determined from amino acid sequence databases and it is possible to locate the above A-region, simply by aligning the sequence of the catalytic unit of the specific kinase chosen, as present in the database, with the PKA-Cα. It is of course desirable to find shorter subsequence of at least 5 continuous amino acids present within this full region, and use these shorter sequences in the candidate compound of the invention.

Finding these short subsequences is a routine procedure, which can be achieved by several possible manners, such as by synthesizing subsequences of 5-10 aa having partially overlapping, or adjacent sequences, and optionally optimizing the chosen sequence (if rather longer sequences such as, for example, 8-10 aa are used) by sequentially deleting from one or both of its terminal amino acids until the optimal shorter sequence. The sequence chosen is not necessarily the shortest, but the best wherein a combination of best activity and shortest sequences are both taken into consideration.

After obtaining shorter subsequence, which still has signal-transduction modulating properties, it is necessary to find which amino acids, either in the sequence of the full region, but preferably in the sequence of the shorter subsequence, are essential (crucial for the modulating activity) and which are non-essential. This can be done by routine procedure, wherein a plurality of sequences are prepared, wherein in each sequence a single (and different) amino acid has been replaced, as compared to the native sequence by a “test amino acid”—usually the amino acid residue Alanine (a procedure known as: “Ala-scan”). Each of the plurality of sequences is again tested for its kinase-associated signal transduction modulating activities. Amino acids which when replaced cause lost, or substantial decrease (statistically significant change) in the modulating activity of the full sequence is considered as “essential amino acids”. Identification of such essential amino asides may be carried out by other SAR techniques such as by site-directed mutagenesis or “omission scan”. Amino acids which when replaced, or omitted, do not caused a statistically significant change of modulating activity of the sequence are referred to as “non-essential” amino acids. A way for determining likely essential amino acids is by determining conserved amino acids among a plurality of kinases (using standard techniques). Such conserved sequences are typically suspected as being essential amino acids.

Finally, as a last step, a plurality of sequences is prepared which may comprise either the full native sequence of the A-region, short subsequence of at least 5 (at least 6, 7, 8, 9, 10, 11, 12, 13, 14, 15, 16 or 17) amino acids as appearing in the A-region, (or the shorter subsequence), sequences wherein at least one essential amino acid has been replaced by conservative substitution by a naturally, non-naturally occurring amino acid or by a peptidomimetic organic moiety; and/or an amino acid sequence wherein at least one amino acid (present in a non-essential position) has been deleted ,or an amino acid in a non-essential position has been replaced by conservative or non-conservative substitution by a naturally occurring, non-naturally occurring, or organic peptidomimetic moiety, and of at least one of any of the above has been chemically modified.

For example, 1, 2, 3, 4, 5, 6, 7, 8, amino acids, both essential and non-essential may be replaced (both by conservative or non-conservative substitution) in the sequence used in a molecule of the invention as compared with the native sequence present in the kinase. The total of replacements should be of no more than 40% of the amino acids, 30% of the amino acid to 20% of the amino acid, or 10% of the amino acid. Preferably, in a short sequence of 10 amino acids there are 3 preferably 2, most preferably 1 non-conservative replacement and 4 preferably 3, more preferably 2, still more preferably 1 conservative replacements so long as the total number of replacements (conservative or non-conservative) in a 10 aa sequence is no more than 4. In longer sequences more replacements can be tolerated and in shorter sequences less replacements are possible.

A notable exception to the above is the use of retro-inverso amino acids (in reverse order as compared to the native sequence), where when the peptide is in the reversed order, all of its amino acids are replaced with their D- counterparts as defined above.

When preparing the compounds, it is possible to proceed by one of two strategies: by one strategy it is possible to test (for kinase-associated signal transduction modulating activities) a full compound—i.e. a molecule comprising both a candidate sequence, and for example, non-amino acid moieties such as hydrophobic moieties present in one of its terminals. This strategy is generally used where the test assay is carried out intact cells or in-vivo where the issue of penetration through membranes, addressed by addition of a hydrophobic moiety, is crucial.

Alternatively, it is possible to first optimize the sequence alone (preferably by testing it in a cell-free system) so as to first find the best A-region sequence variant, or shortest A-region subsequence possible, and then add to the chosen sequence other moieties, such as hydrophobic moieties, etc. to improve other properties of the compound, such as penetration to ceils, resistance to degradation, etc.

The present invention also concerns compounds for the modulation of signal transduction associated kinase obtained by the above methods.

The present invention also concerns a compound which has the property of modulation of signal transduction of a kinase comprising of at least one moiety for transport across cellular membranes, in association with a sequence selected from:

(1) a sequence comprising of from a minimum of 5 continuous amino acids of said A-region to a maximum of all the continuous amino acids of said A-region;

(2) a variant of the sequence of (1) wherein up to 40% of the amino acids of the sequence of (1) have been replaced with a naturally or non-naturally occurring amino acid or with a peptidomimetic organic moiety; and/or up to 40% of the amino acids have their side chains chemically modified, and/or up to 20% of the amino acids have been deleted, provided that at least 50% of the amino acids of (1) are maintained unaltered in the variant;

(3) a sequence of (1) or (2) wherein one or more of the amino acids is replaced by the corresponding D-amino acid;

(4) a sequence of any one of (1) to (3) wherein at least one peptidic backbone atom, or peptidic backbone bond has been altered to a modified peptidic backbone atom or a non-naturally occurring peptidic backbone bond, respectively;

(5) a sequence of any one of (1), (2), (3) or (4) in a reverse order; and

(6) a combination of two or more of the sequences of (1), (2), (3), (4) or (5).

The term “moiety for transport across cellular membranes” refers to a chemical entity, or a composition of matter (comprising several entities) that causes the transport of members “associated” (see below) with it through phospholipdic membranes. One example of such moieties are linear, branched, cyclic, polycyclic or hetrocyclic substituted or non-substituted hydrocarbons. Another example of such a moiety are short peptides that cause transport of molecules attached to them into the cell by, gradient derived, active or facilitated transport, as well as other non-peptidic moieties known to be transported through membranes such as glycosylated steroid derivatives, and the like. Other examples are moieties known to be internalized by receptors such as EGF, or trasfferin agonists. The moiety of the compound may be a polymer, liposome or micelle containing, entrapping or incorporating therein the amino acid sequence. In such a case the compound is the polymer, liposome micelle etc impregnated with the amino acid sequence.

The term “in association” concerns covalent binding both of the type that is relatively permanent and of the type that can be cleaved by enzymes. The term may include entrapment (inside liposome), impregnation (in polymers), complexion through salt formation which can be dissociated in specific pH, or a specific ionic concentration.

The present invention further concerns pharmaceutical compositions comprising the above compounds as active ingredients. The pharmaceutical composition may contain one species of the compound of the invention or a combination of several species of the compounds of the invention.

The pharmaceutical compositions of the invention should be used for treatment of conditions or disorders wherein a therapeutical beneficial effect can be evident through the modulation of kinase-associated signal transduction.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIGS. 1A-1B are a table illustrating the amino acid sequences of the A region of the following protein kinases: Src, Yes, Fyn, Fgr, Lyn, Hck, Lck (SEQ ID NO. 1 to 7); Csk and Matk (SEQ ID NO. 8 to 9); focal adhesion kinase (FAK) (SEQ ID NO. 10); c-Ab1 (SEQ ID NO. 11); endothelial growth factor receptors Tie, Tek, FGF receptor (Bek, Flg, FGFR3, FGFR4), PDGF receptor a and b, Flt 1 and 4 and Flk1 (SEQ ID NO. 12 to 19); HGF receptors c-Met, c-Sea and Ron (SEQ ID NO. 20 to 22); EGF receptor (EGFR, ErbB2, ErbB3, ErbB4) (SEQ ID NO. 23 to 26); Ret (SEQ ID NO. 27); NGF receptors (Trk) (SEQ ID NO. 28 to 29); Syk and Zap70 (SEQ ID NO. 30 to 31); Jak kinases 1 through 3 and Tyk2 (SEQ ID NO. 32 to 35); insulin receptor kinase (IRK) (SEQ ID NO. 36); Activin receptor-like kinases 1 through 6 (ALK1, 2, 3, 4, 5, 6) (SEQ ID NO. 37 to 40); discoidin domain receptors 1 and 2 (DDR) (SEQ ID NO. 41 to 42); ACK (SEQ ID NO. 43); Ephrin receptor B4 (SEQ ID NO. 44); TEC (SEQ ID NO. 45); Polo family kinases Plk, Plx1, polo, SNK, CDC5, Sak, Prk, Fnk and Plo1 (SEQ ID NO.46 to 53).

FIGS. 2A-2E are a group of sequences illustrating the consensus amino acid sequences of the A region found among the family of protein kinases. Also shown are examples of conservative substitutions in these amino acid sequences. An “*” indicates an aliphatic, substituted aliphatic, benzylic, substituted benzylic, aromatic or substituted aromatic ester of glutamic acid or aspartic acid.

FIGS. 3A-3B are a Table illustrating the sequences of the following compounds: Plk K035A100; Plx1 K036A100; polo K037A100; SNK K038A100; CDC5 K039A100; Sak K040A100; Prk K041A100; Plo1 K043A100; ALK1 K048A100; c-Src K051A100; c-Yes K052A100; Fyn K053A100; c-Fgr K054A100; Lyn K055A100; Hck K056A100; Lck K057A100; Csk K058A100; Matk K059A100; Fak K060A100; c-Ab1 K061A100; Tie K062A100; PDGFR-b K064A100; PDGFR-a K065A100; Flt1 K066A100; Flt4 K067A100; Flg K069A100; FGFR-4 K072A100; c-Met K073A100; c-Sea K074A100; Ron K075A100; EGFR K076A100; ErbB2 K077A100; ErbB3 K078A100; ErbB4 K079A100; Ret K080A100; Trk-NGFR K081A101 K081A102 K081A103 K0; Syk K082A100; Zap70 K083A100; Jak1 K084A100; Jak2 K085A100; Jak3 K086A100; IRK K094A103 K094A104 K094A105 K094A106 K094A107 K094A108 K094A112 K094A113 K094A114 K094A115 K094A116 K094A118 K094A119 K094A131 K094A132 K094A122; ALK2 K097A100; ALK3 K098A100; TrkB K102A100; DDR1 K104A100; DDR2 K105A100; Tyk2 K108A100; Eph-B4 K114A100; ITK/TSK K140A100; ACK K141A100 (SEQ ID NO. 54 to 122, respectively).

Peptides are N-myristylated and C-amidated. “K+” indicates a benzoylated lysine residue (epsilon amino). “C5” indicates a lysine-epsilon-amino cysteine. “C6” indicates an alanine-beta-amino cysteine. FIG. 3 shows that one or more glycine residues can be added to the N-terminus of the native A-region amino acid sequence. FIG. 3 also indicates from which protein kinase each peptide is derived.

FIG. 4 shows the result of “Ala-scan” as determined by glucose uptake assay (Example 2).

FIG. 5A shows the 3D structure of IRK in a space-filled manner, and FIG. 5B in a “stick” manner, colored by accessibility (dark to light from the most to the least accessible):.

FIG. 6A shows glucose uptake in the presence of two compounds of the invention (“107”, “205”) alone or in combination with 10 μU insulin; and FIG. 6B shows glucose uptake in the presence of a different concentrations of the compound (“107”) of the invention.

FIG. 7 shows the blood glucose levels in an animal model of diabetes Type I, after administration with two compounds of the invention.

FIG. 8 shows the effect of a compound of the invention in the neuronal crest migration in the presence or absence of noggin.

FIG. 9. shows western blot showing increase of phosphorylation of IRK and PKB in the presence of the compound comprising an IRK derived peptide of the invention and insulin.

FIG. 10 shows western blot indicating decrease of phosphorylation of IRK substrates, in a dose dependent manner in the presence of the compound of the invention (618 derived from an A-region) and lack of effect in the presence of a control compound not derived from the A-region.

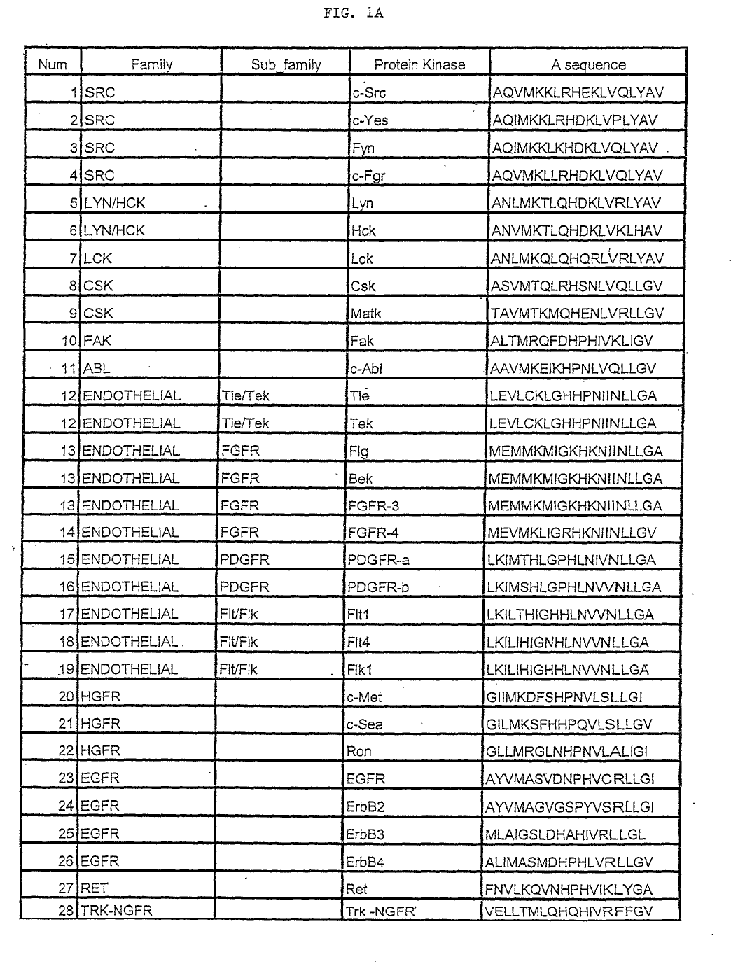

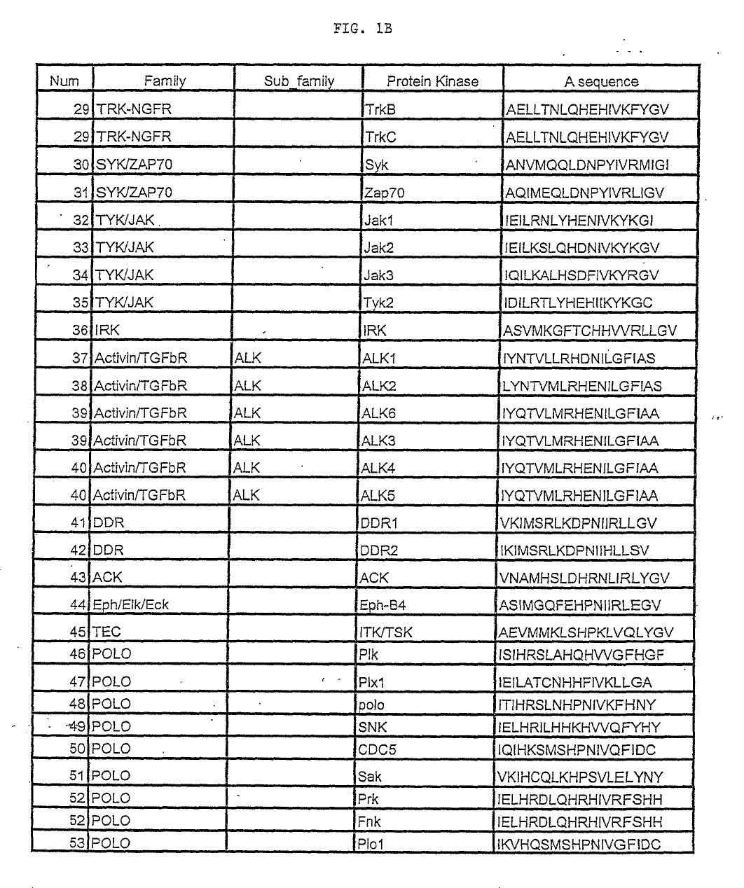

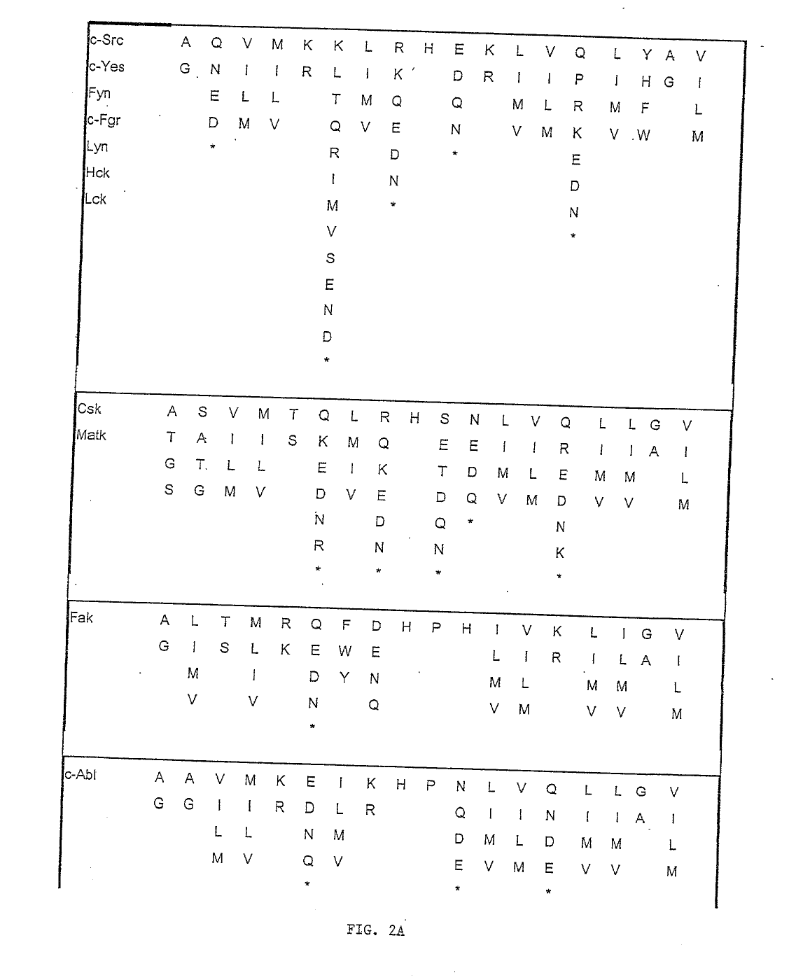

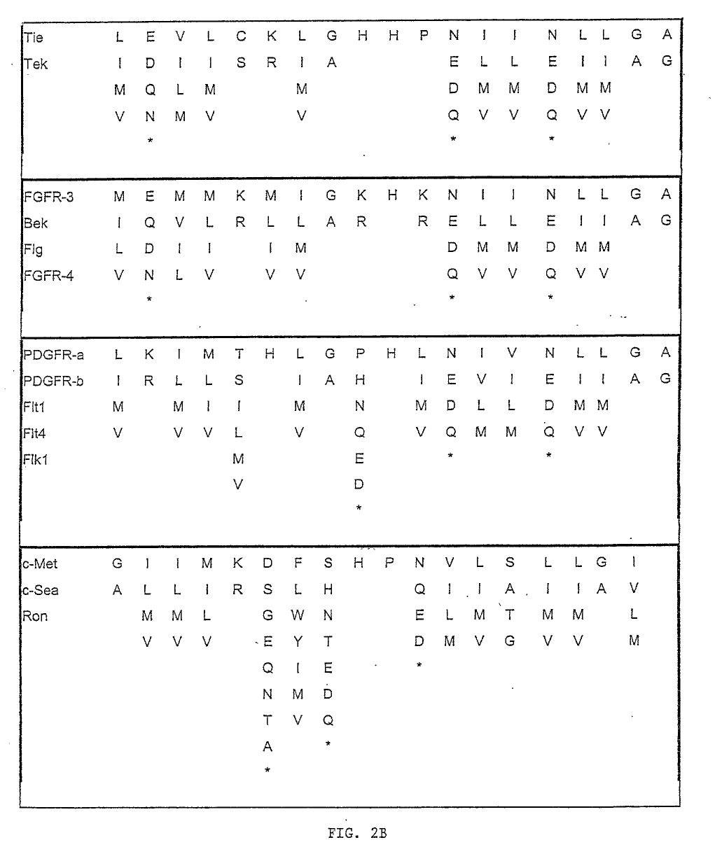

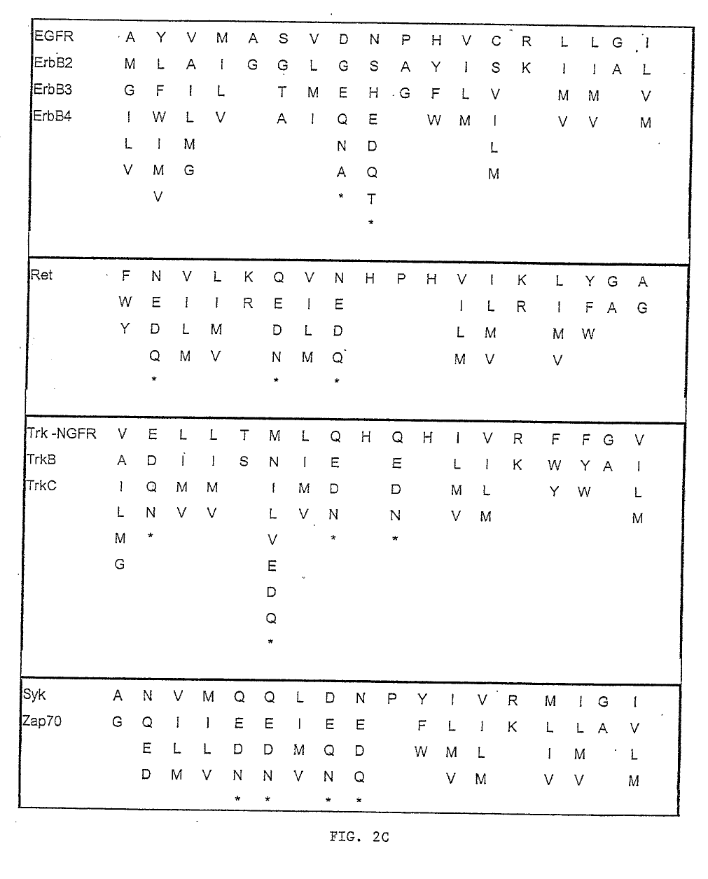

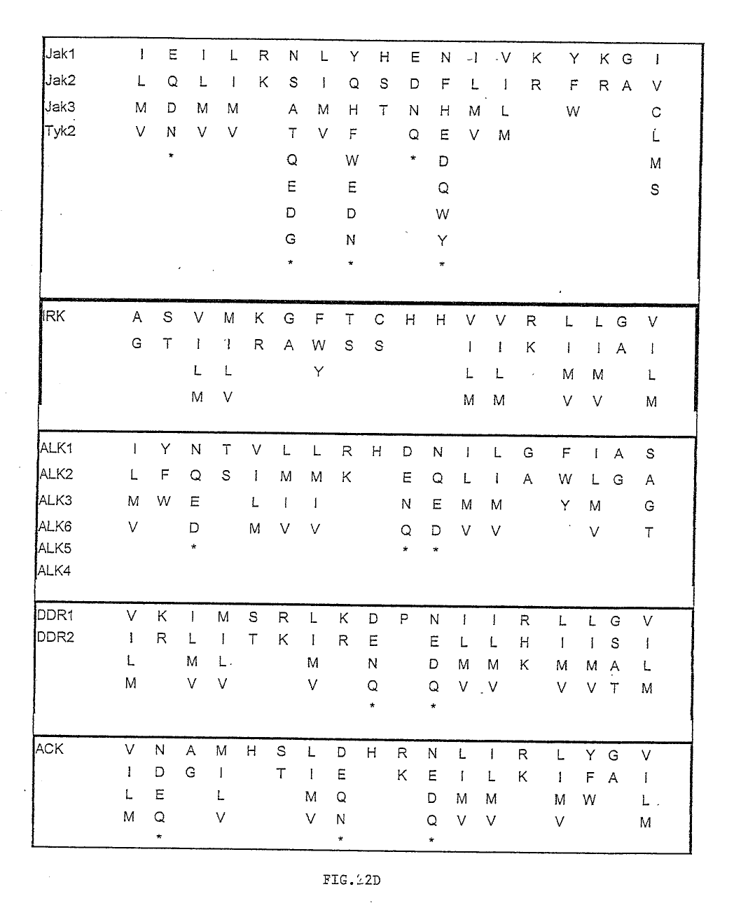

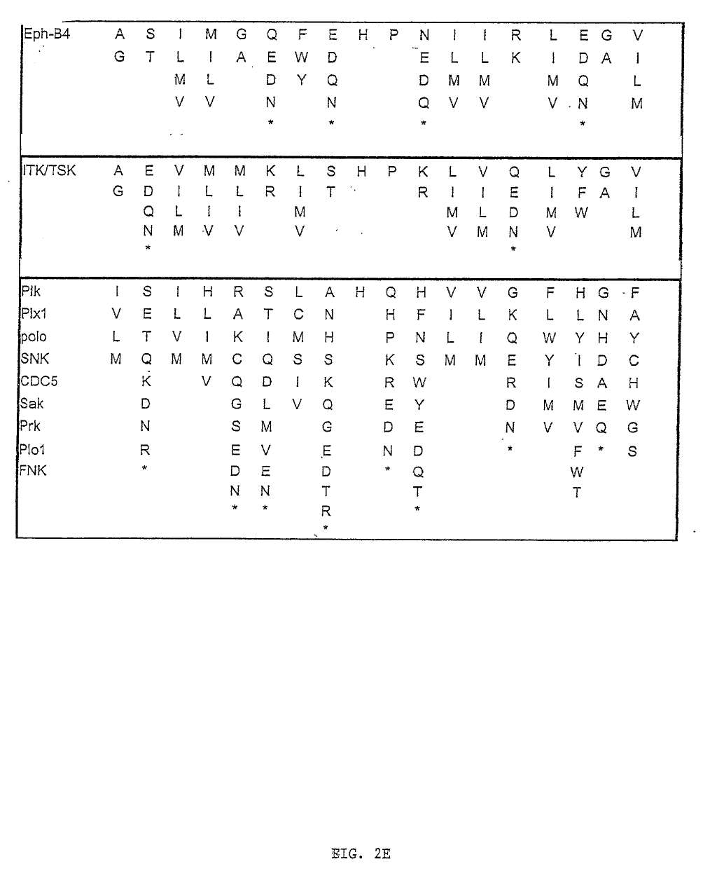

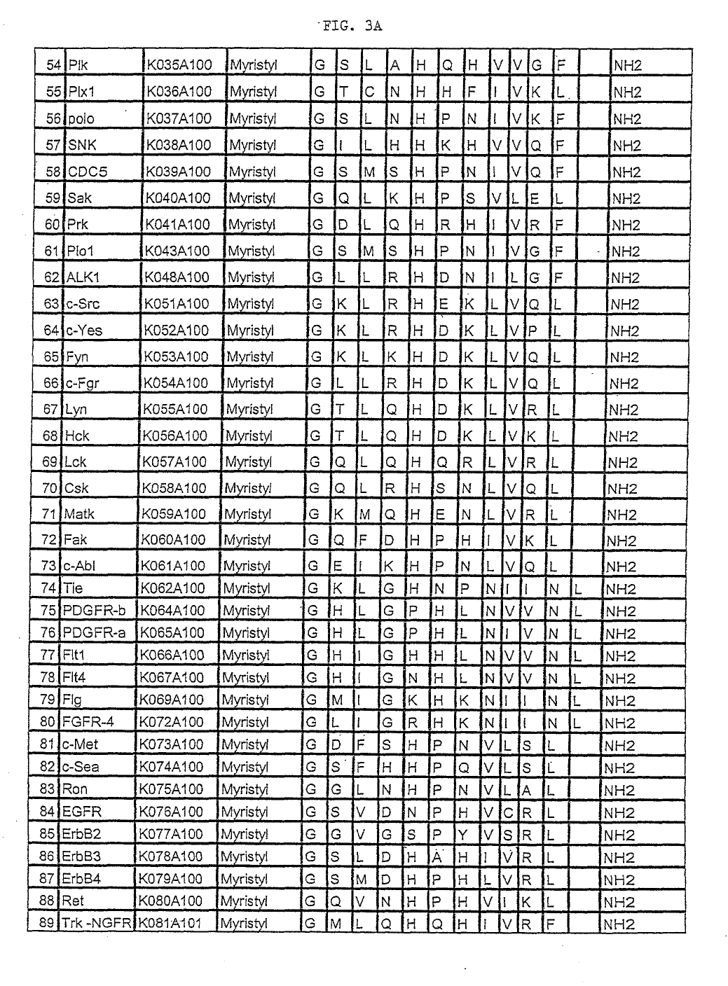

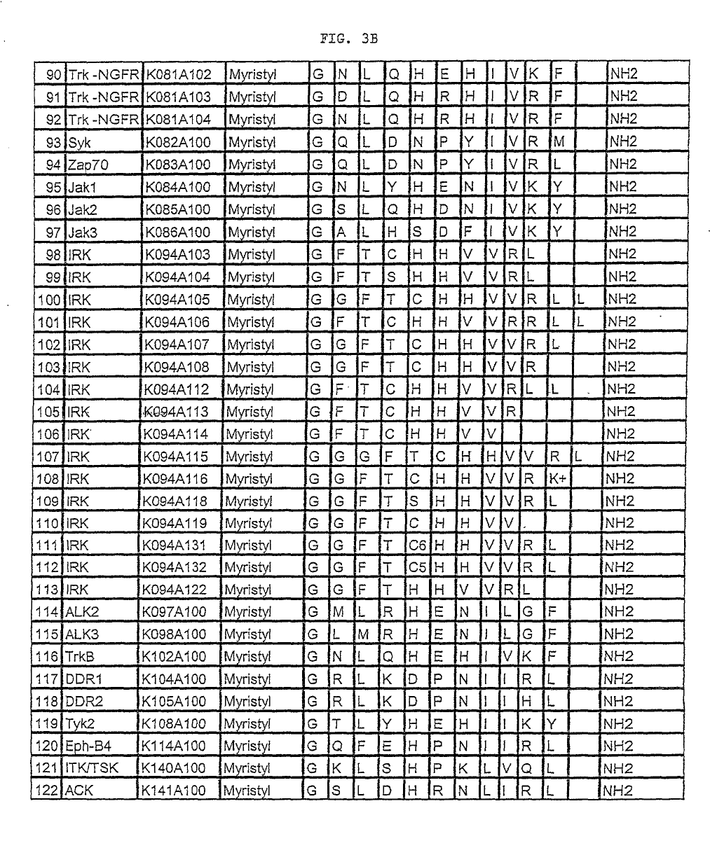

### DETAILED DESCRIPTION OF THE INVENTION

1. Addition of Non-peptidic Groups to one or to Both of the Terminals of the A-derived Sequences to Produce the Compound of the Invention

Where the compound of the invention is a linear molecule, it is possible to place in any of its terminals various functional groups. The purpose of such a functional group may be for the improvement of the modulating activities of the kinase associated signal transduction. The functional groups may also serve for the purpose of improving physiological properties of the compound not related directly to signal transduction modulation properties such as: improvement in stability, penetration, tissue localization, efficacy, decreased clearance, decreased toxicity, improved selectivity, improved resistance to repletion by cellular pumps, improved, or existence of penetration through barriers (blood-brain, gut) and the like. For convenience sake the free N-terminal of one of the sequences contained in the compounds of the invention will be termed as the N-terminal of the compound, and the free C-terminal of the sequence will be considered as the C-terminal of the compound (these terms being used for convenience sake). Either the C-terminus or the N-terminus of the sequences, or both, can be linked to a carboxylic acid functional groups or an amine functional group, respectively.

Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds these being an example for “a moiety for transport across cellular membranes ”.

These moieties can be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al., Arch. Biochem. Biophys. 239:538 (1985) and Singhal et al., FASEB J 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a compound of the present invention is protected, preferably with a methyl, ethyl, benzyl or substituted benzyl ester, more preferably as a benzyl ester.

In addition, a modified lysine residue can be added to the C-terminal of the compound to enhance biological activity. Examples of lysine modification include the addition of an aromatic substitute, such as benzoyl benzoic acid, dansyl-lysine various derivatives of benzoic acids (difluoro-, trifluromethy-, acetamido-, dimethyl-, dimethylamino-, methoxy-) or various derivatives of carboxylic acid (pyrazine-, thiophene-, pyridine-, indole-, naphthalene-, biphenyl,), or an aliphatic group, such as acyl, or a myristic or stearic acid, at the epsilon amino group of the lysine residue.

Examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO—and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO—and benzyl-O—CO—, (substituted benzyl)-O—CO—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the molecule can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH_{2,} —NHCH₃, —N(CH₃)₂, —NH(ethyl), —N(ethyl)₂, —N(methyl) (ethyl), —NH(benzyl), —N(C1-C4 alkyl)(benzyl), —NH(phenyl), —N(C1-C4 alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

Preferably the compounds includes in the N-terminal a hydrocarbon having a length of C₄-C₁₂ preferably C₆-C₁₈, most preferably C₁₀-C₁₆. Example of hydrophobic moieties are: aaystyl, stearyl, lauroyl, palmitoyl and acetyl etc.

2. Variants and Short Sequences

As one of the main mechanisms of action of the amino acid portion of the compounds of the invention is interruption of peptide-peptide interaction, it is clear that for such an interruption it is possible to use as a mimic one of several short sequences in the region. In addition, the mimic does not have to be identical to the sequence in the region since for the purpose of interruption (at least 50%) similarity is required but a 100% is not a pre-requisite as will be shown below.

3. Finding a Shorter Subsequences of the A-region

As indicated, the A-region from which the continuous stretch of at least five amino acids is chosen is identified by aligning the amino acid of the catalytic unit of a specific kinase, involved in the specific kinase-associated signal transduction to be modulated, with the catalytic unit of PKA-Cα and determining the positions corresponding to 92-109 (in the actual kinase chosen the sequence may be longer or shorter than 18 aa, as typically, alignment programs can identify such missing or additional amino acids in the kinase as compared to the PKA-Cα).

A shorter subsequence of the A-region comprising a continuous stretch of at least five amino acid can be found by preparing a series of partially overlapping peptides each of 5-10 amino acids and each obtained by synthesizing a sequence that is one position removed from the previous sequence.

For example, if the A-region of a specific kinase is in position 200-218, (in this case 19 aa long since at times the A-region is more than 18 aa) and it is to be desired to prepare 10 aa peptides, then the following, partially overlapping peptides are prepared, a peptide having the sequence 200-209, 201-210, 20 2-211, . . . 209-218. The kinase-associated signal transduction activity is then determined in a test assay. The best 10-aa peptide is then chosen.

For checking whether the 10 aa peptide can be reduced in sequence, it is possible to either repeat the above procedure (preparing a series of partially overlapping peptides) using 5 aa long peptides that span the length of the 10 aa peptide, or to shorten the 10 aa peptide by deleting alternatively from each terminal, an amino acid, and testing the kinase-associated signal transduction modulating activity of the progressively truncated peptides, until the optimal sequence of at least 5, at least 6, at least 7, at least 8, at least 9 aa peptide is obtained or determining whether longer sequences are required. For example, in a specific signal transduction associated by the kinase it is possible that the 10 aa are the shortest region possible. As the A-region is relatively small, typically no longer than 18-25 aa (in some kinases), the number of different peptides to be tested is also small. For example, for an A-region having a length of about 20 aa, there is a need to prepare only 12 peptides to find the optimal 8 aa peptide. After the best 8-aa peptide is obtained, it is possible to delete sequentially amino acids from one or both terminals of the 8 per peptide for obtaining the shortest sequence of 5, 6 or 7 aa that is still active. For these steps only 16 sequences have to be tested, so that by testing only 24 peptides it is possible to find such a shorter sequence. Typically the peptide is 5-15, preferably 7-13 amino acids long.

4. Identifying Essential and Non-essential Amino Acids in the Subsequence Chosen

A. Ala-scan

Once the shorter continuous stretch of at least 5 (at least 6, 7, 8, 9, 10, 11 or 12) amino acids has been identified, as explained above, it is necessary to realize which of the amino acids in the stretch are essential (i.e. crucial for the kinase-associated signal transduction modulation) and which are non-essential. Without wishing to be bound by theory, in almost every native protein involved in interaction with other cellular components, some amino acids are involved with the interaction (essential amino acids) and some amino acids are not involved in the interaction (non-essential amino acids), for example since they are cryptic. A short peptide which is to mimic a region of the kinase protein behaves in the same way as the region when present in the full kinase: some amino acids actually interact with the substrate (or other interacting components) and other amino acids merely serve to spatially position the interacting amino acids, but do not participate in the interaction with the other cellular components.

Essential amino acids have to be maintained (i.e. be identical to those appearing in the native kinase), or replaced by conservative substitutions (see definition below) to obtain variants of the peptides. Non-essential amino acids can be deleted, or replaced by a spacer or by conservative or non-conservative substitutions.

Identification of essential vs. non-essential amino acids in the peptide can be achieved by preparing several peptides that have a shorter sequence (see 2 above) in which each position is sequentially replaced by the amino acid Ala (“Ala-Scan. ”). This allows to identify the amino acids which modulating activity is decreased by said replacement (“essential”) and which are not decreased by said substitution (“non-essential”) (Morrison et al., Chemical Biology 5:302-307, 2001). Another option for testing the importance of various peptides is by the use of site-directed mutagenesis.

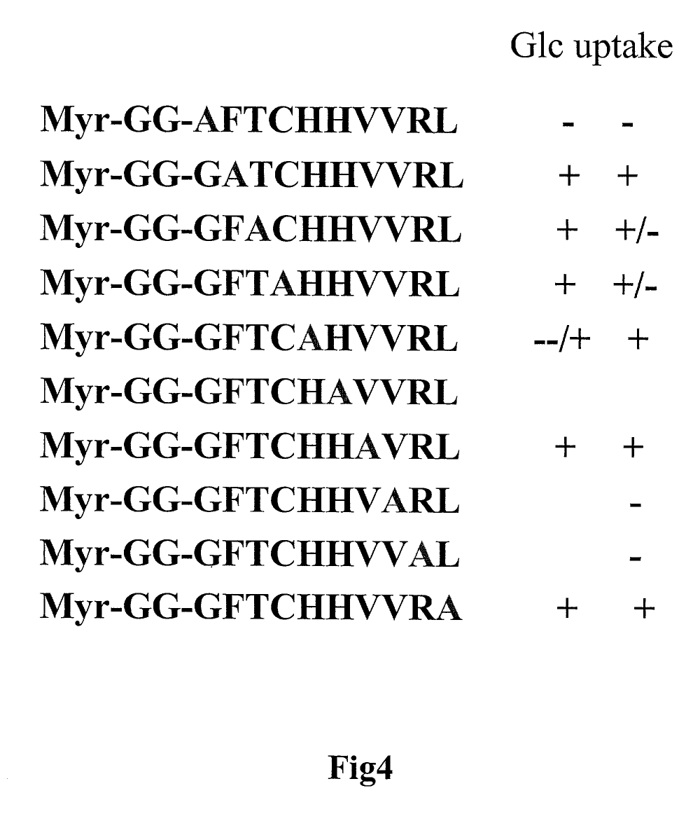

FIG. 4 shows the results of such an Ala-scan when each of the amino acids in the A-region of IRK was sequentially replaced by Alanine, and the compound comprising myristyl-GG conjugated to the Ala-containing sequence was tested in a glucose uptake assay. (+effective in glucose uptake−non-effective, ±effective only in very high concentrations). As can be seen the amino acid G, V and R are essential (when replaced causes loss or decrease of glucose uptake activities) and the remaining aa are non-essential.

B. 3D-analysis

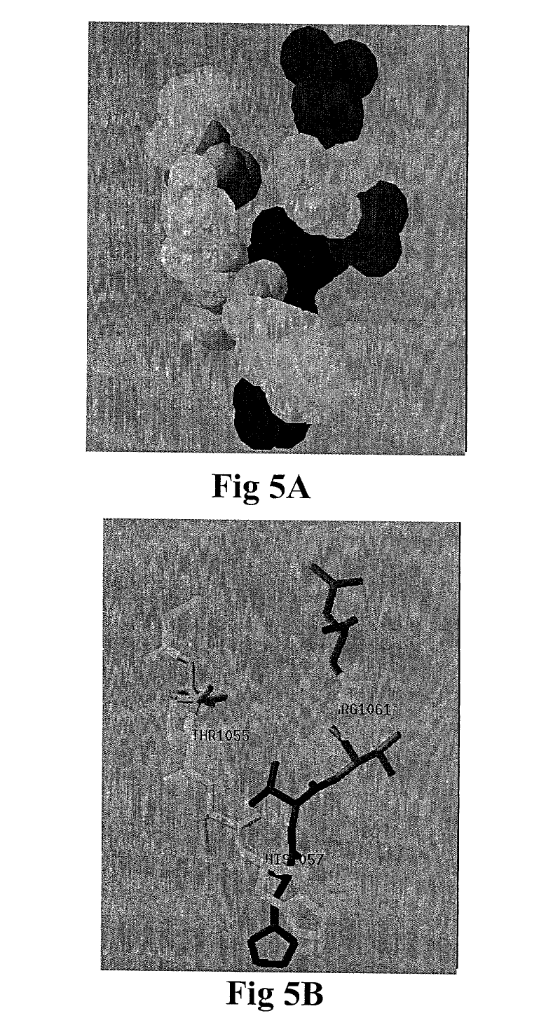

Another strategy for finding essential vs. non-essential amino acids is by determining which aa of the A-region, in the 3D of the full kinase are exposed and which are cryptic. FIGS. 5A and 5B show the 3D structure of the A-region (when determined as a part of the full kinase) where the degree of exposure is determined by coloring (dark cryptic, lighter more exposed ). Typical cryptic aa are non-essential (exposed or partially exposed are more likely to be essential). However, if one wishing to “guess” theoretically which “non-conservative” substitutions in the cryptic region can be tolerated, a good guideline is to “check” on a 3D computer model of the full kinase, when the peptide is superimposed on the kinase in the exact position from which the native sequence was obtained, whether these changes drastically alter the overall shape of the A-regions. Those non-conservative substitutes, that when simulated on a computer 3D structure (for example using the Triphase™ software) do not cause drastic after action of the overall steps of the A-region (drastic shifting in the position of the exposed aa) are likely non-conservative replacements. Thus prior to experimental testing it is possible to reduce the number of tested candidates by computer simulation. Where the 3D structure of a specific kinase is not available in activating crystallography data, it is possible to obtain a “virtual” 3D structure of the kinase based on homology to known crystallographic structures using such progress such as Compser™ (Tripose, USA).

5. Obtaining Variants

The sequence regions of the compound of the invention may be the native sequences obtained from the kinase (preferably the shortest possible sequence from the region that has the highest activity), or alternatively variants of the native sequence obtained by deletion, (of non-essential amino acids) or substitution (only conservative substitutions in essential positions, both conservative and non-conservative of non-essential acids). As well as by chemical modifications of the side chains.

5.1 Deletions and Insertions

Deletions can occur in particular of the “non-essential amino acids”. Additions may occur in particular at the N-terminal or the C-terminal of any of the amino acids of the sequence. Insertions should preferably be N-terminal or C-terminal to the sequence of (b1) to (b5) or between the several sequences linked to each other (b6). However other insertions or deletions are possible.

5.2 Replacements

The variants can be obtained by replacement (termed also in the text as “substitution ”) of any of the amino acids as present in the native kinase. As may be appreciated there are positions in the sequence that are more tolerant to substitutions than others, and in fact some substitutions may improve the activity of the native sequence. The determination of the positions may be realized using Ala-Scan, “omission scan” “site directed mutagenesis” as described and 3D theoretically considerations in 3 above. Generally speaking the amino acids which were found to be “essential” should either be identical to the amino acids present in the native specific kinase or alternatively substituted by “conservative substitutions” (see below). The amino acids which were found to be “non-essential” might be identical to those in the native peptide, may be substituted by conservative or non-conservative substitutions, and may be deleted or replaced by a “spacers”.

The term “naturally occurring amino acid” refers to a moiety found within a peptide and is represented by —NH—CHR—CO—, wherein R is the side chain of a naturally occurring amino acid.

The term “non-naturally occurring amino acid” (amino acid analog) is either a peptidomimetic and D- counterpart of a naturally occurring amino acid, or is a D or L residue having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. This term also refers to the D-amino acid counterpart of naturally occurring amino acids. Amino acid analogs are well-known in the art; a large number of these analogs are commercially available. Many times the use of non- naturally occurring amino acids in the peptide has the advantage that the peptide is more resistant to degradation.

The term “conservative substitution” in the context of the present invention refers to the replacement of an amino acid present in the native sequence in the specific kinase with a naturally or non-naturally occurring amino or a peptidomimetic having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid). However where the native amino acid to be replaced is charged, the conservative substitution according to the definition of the invention may be with a naturally occurring amino acid, a non-naturally occurring amino acid or a peptidomimetic moiety which are charged, or with non-charged (polar, hydrophobic) amino acids that have the same steric properties as the side-chains of the replaced amino acids. The purpose of such a procedure of maintaining the steric properties but decreasing the charge is to decrease the total charge of the compound.

For example in accordance with the invention the following substitutions are considered as conservative: replacement of arginine by cytroline; arginine by glutamine; aspartate by asparagine; glutamate by glutamine.

As the naturally occurring amino acids are grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid would have the same or a similar functional group in the side chain as the original amino acid.

The following are some non-limiting examples of groups of naturally occurring amino acids or of amino acid analogs are listed bellow. Replacement of one member in the group by another member of the group will be considered herein as conservative substitutions:

Group I includes leucine, isoleucine, valine, methionine, phenylalanine, serine, cysteine, threonine and modified amino acids having the following side chains: ethyl, n-butyl, —CH₂CH₂OH, —CH₂CH₂CH₂OH, —CH₂CHOHCH₃ and —CH₂SCH₃. Preferably Group I includes leucine, isoleucine, valine and methionine.

Group II includes glycine, alanine, valine, serine, cysteine, threonine and a modified amino acid having an ethyl side chain. Preferably Group II includes glycine and alanine.

Group III includes phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, methoxy, ethoxy and —CN. Preferably, Group III includes phenylalanine, tyrosine and tryptophan.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, CO—NH-alkylated glutamine or asparagine (e.g., methyl, ethyl, n-propyl and iso-propyl) and modified amino acids having the side chain —(CH₂)_{3—}COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, glutamine, asparagine, methyl aspartate, ethyl aspartate, benzyl aspartate and methyl glutamate, ethyl glutamate and benzyl glutamate.

Group V includes histidine, lysine, arginine, N-nitroarginine, β-cycloarginine, μ-hydroxyarginine, N-amidinocitruline and 2-amino-4-guanidinobutanoic acid, homologs of lysine, homologs of arginine and ornithine. Preferably, Group V includes histidine, lysine, arginine, and ornithine. A homolog of an amino acid includes from 1 to about 3 additional methylene units in the side chain.

Group VI includes serine, threonine, cysteine and modified amino acids having C1-C5 straight or branched alkyl side chains substituted with —OH or —SH. Preferably, Group VI includes serine, cysteine or threonine.

In this invention any cysteine in the original sequence or subsequence can be replaced by a homocysteine or other sulfhydryl-containing amino acid residue or analog. Such analogs include lysine or beta amino alanine, to which a cysteine residue is attached through the secondary amine yielding lysine-epsilon amino cysteine or alanine-beta amino cysteine, respectively.

The term “non-conservative substitutions” concerns replacement of the amino acid as present in the native kinase by another naturally or non-naturally occurring amino acid, having different electrochemical and/or steric properties, for example as determined by the fact the replacing amino acid is not in the same group as the replaced amino acid of the native kinase sequence. Those non-conservative substitutions which fall under the scope of the present invention are those which still constitute a compound having kinase-associated signal transduction modulating activities. Because D-amino acids have hydrogen at a position identical to the glycine hydrogen side-chain, D-amino acids or their analogs can often be substituted for glycine residues, and are a preferred non-conservative substitution

A “non-conservative substitution” is a substitution in which the substituting amino acid (naturally occurring or modified) has significantly different size, configuration and/or electronic properties compared with the amino acid being substituted. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)₅₋COOH]—CO— for aspartic acid.

Alternatively, a functional group may be added to the side chain, deleted from the side chain or exchanged with another functional group. Examples of non-conservative substitutions of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. In yet another alternative, the side chain of the substituting amino acid can have significantly different steric and electronic properties from the functional group of the amino acid being substituted. Examples of such modifications include tryptophan for glycine, lysine for aspartic acid and —(CH₂)₄COOH for the side chain of serine. These examples are not meant to be limiting.

As indicated above the non-conservative substitutions should be of the “non-essential” amino acids.

“Peptidomimetic organic moiety” can be substituted for amino acid residues in the compounds of this invention both as conservative and as non-conservative substitutions. These peptidomimetic organic moieties either replace amino acid residues of essential and non-essential amino acids or act as spacer groups within the peptides in lieu of deleted amino acids (of non-essential amino acids). The peptidomimetic organic moieties often have steric, electronic or configurational properties similar to the replaced amino acid and such peptidomimetics are used to replace amino acids in the essential positions, and are considered conservative substitutions. However such similarities are not necessarily required. The only restriction on the use of peptidomimetics is that the compounds retain their tissue-remodeling modulating activity as compared to compounds constituting sequence regions identical to those appearing in the native kinase.

Peptidomimetics are often used to inhibit degradation of the peptides by enzymatic or other degradative processes. The peptidomimetics can be produced by organic synthetic techniques. Examples of suitable peptidomimetics include D amino acids of the corresponding L amino acids, tetrazol (Zabrocki et al., J. Am. Chem. Soc. 110:5875-5880 (1988)); isosteres of amide bonds (Jones et al., Tetrahedron Lett. 29: 3853-3856 (1988));

LL-3-amino-2-propenidone-6-carboxylic acid (LL-Acp) (Kemp et al., J. Org. Chem. 50:5834-5838 (1985)). Similar analogs are shown in Kemp et al., Tetrahedron Lett. 29:5081-5082 (1988) as well as Kemp et al., Tetrahedron Lett. 29:5057-5060 (1988), Kemp et al., Tetrahedron Lett. 29:4935-4938 (1988) and Kemp et al., J. Org. Chem. 54:109-115 (1987). Other suitable peptidomimetics are shown in Nagai and Sato, Tetrahedron Lett. 26:647-650 (1985); Di Maio et al., J. Chem. Soc. Perkin Trans., 1687 (1985); Kahn et al., Tetrahedron Lett. 30:2317 (1989); Olson et al., J. Am. Chem. Soc. 112:323-333 (1990); Garvey et al., J. Org. Chem. 56:436 (1990). Further suitable peptidomimetics include hydroxy-1,2,3,4-tetrahydroisoquinoline-3-carboxylate (Miyake et al., J. Takeda Res. Labs 43:53-76 (1989)); 1,2,3,4-tetrahydro-isoquinoline-3-carboxylate (Kazmierski et al., J. Am. Chem. Soc. 133:2275-2283 (1991)); histidine isoquinolone carboxylic acid (HIC) (Zechel et al., Int. J. Pep. Protein Res. 43 (1991)); (2S, 3S)-methyl-phenylalanine, (2S, 3R)-methyl-phenylalanine, (2R, 3S)-methyl-phenylalanine and (2R, 3R)-methyl-phenylalanine (Kazmierski and Hruby, Tetrahedron Lett. (1991)).

5.3 Chemical Modifications

In the present invention the side chains of the amino acid residue appearing in the native sequence may be chemically modified when the individual residue is isolated, and that the chemically modified amino acid residue may be used as a building block, in the process of synthesis of the molecule, i.e. during elongation of the amino acid chain. Another alternative is chemical modification of an amino acid when it is present in the molecule or sequence (“in situ” modification).

The amino acid of any of the sequence regions of the molecule can be chemically modified by carboxymethylation, carboxyacrylation, acylation, phosphorylation, iodination, glycosylation or fatty acylation. Ether bonds can be used to join the serine or threonine hydroxyl to the hydroxyl of a sugar. Amide bonds can be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also be formed between amino acids and carbohydrates. Fatty acid acyl derivatives can be made, for example, by free amino group (e.g., lysine) acylation (Toth et al., Peptides: Chemistry, Structure and Biology, Rivier and Marshal, eds., ESCOM Publ., Leiden, 1078-1079 (1990)). Additions of various groups to Lysine residue are also disclosed above.

5.4 Cyclization of the molecule

The present invention also includes cyclic compounds which are cyclic molecules.

A “cyclic molecule ” refers, in one instance, to a compound of the invention in which a ring is formed by the formation of a peptide bond between the nitrogen atom at the N-terminus and the carbonyl carbon at the C-terminus.

“Cyclized” also refers to the forming of a ring by a covalent bond between the nitrogen at the N-terminus of the compound and the side chain of a suitable amino acid in the sequence present therein, preferably the side chain of the C-terminal amino acid. For example, an amide can be formed between the nitrogen atom at the N-terminus and the carbonyl carbon in the side chain of an aspartic acid or a glutamic acid. Alternatively, the compound can be cyclized by forming a covalent bond between the carbonyl at the C-terminus of the compound and the side chain of a suitable amino acid in the sequence contained therein, preferably the side chain of the N-terminal amino acid. For example, an amide can be formed between the carbonyl carbon at the C-terminus and the amino nitrogen atom in the side chain of a lysine or an ornithine. Additionally, the compound can be cyclized by forming an ester between the carbonyl carbon at the C-terminus and the hydroxyl oxygen atom in the side chain of a serine or a threonine.

“Cyclized” also refers to forming a ring by a covalent bond between the side chains of two suitable amino acids in the sequence present in the compound, preferably the side chains of the two terminal amino acids. For example, a disulfide can be formed between the sulfur atoms in the side chains of two cysteines. Alternatively, an ester can be formed between the carbonyl carbon in the side chain of, for example, a glutamic acid or an aspartic acid, and the oxygen atom in the side chain of, for example, a serine or a threonine. An amide can be formed between the carbonyl carbon in the side chain of, for example, a glutamic acid or an aspartic acid, and the amino nitrogen in the side chain of, for example, a lysine or an ornithine.

In addition, a compound can be cyclized with a linking group between the two termini, between one terminus and the side chain of an amino acid in the compound, or between the side chains to two amino acids in the peptide or peptide derivative. Suitable linking groups are disclosed in Lobl et al., WO 92/00995 and Chiang et al., WO 94/15958, the teachings of which are incorporated into this application by reference.

6. Pharmaceutical Compositions and Therapeutical Methods of Treatment

The compound of the present invention can be used as an active ingredient (together with a pharmaceutically acceptable carrier) to produce a pharmaceutical composition. The pharmaceutical composition may comprise one, or a mixture of two or more of the compounds of the invention in an acceptable carrier. A combination of two or more different compounds is desirable for example, where a disease or condition requires the modulation of two or more kinase-associated signaling (either in the same or in different pathways) In such a case the composition may comprise two different compounds, each comprising a sequence derived from the A-region of a different kinase.

The pharmaceutical composition should be used for the treatment of a disease disorder or pathological condition wherein a therapeutically beneficial effect may be evident due to modulation (increase or decrease) of at least one kinase-associated signal transduction. Typically those are diseases in which one of their manifestations (a manifestation that may be the cause or the result of the disease) is non-normal kinase-associated signaling transduction, or diseases or conditions where, although the activity is normal, a therapeutical beneficial effect may nonetheless be evident by modulating (increasing or decreasing) the activity of the kinase-associated signal transduction (for example elimination of scarring which is a natural consequence of wound healing). Examples of such disease are selected from: cancer, restenosis, atherosclerosis, psoriasis, arthritis, benign prostatic hypertrophy, autoimmune diseases, osteoporosis, septic shock, diabetics as well as conditions diseases and disorders involving tissue remodeling.

The compounds of the present invention can be administered parenterally. Parenteral administration can include, for example, systemic administration, such as by intramuscular, intravenous, subcutaneous, or intraperitoneal injection. Compounds which resist proteolysis can be administered orally, for example, in capsules, suspensions or tablets. The compound can also be administered by inhalation or insufflations or via a nasal spray.

The compound can be administered to the individual in conjunction with an acceptable pharmaceutical carrier as part of a pharmaceutical composition for treating the diseases discussed above. Suitable pharmaceutical carriers may contain inert ingredients which do not interact with the compounds. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., Controlled Release of Biological Active Agents, John Wiley and Sons, 1986). The formation may be also resources for administration to bone, or in the form of salve, solution, ointment, etc. for topical administration.

7. Preparation of the Compounds

Peptide sequences for producing any of the sequence of the compounds of the invention may be synthesized by solid phase peptide synthesis (e.g., t-BOC or F-MOC) method, by solution phase synthesis, or by other suitable techniques including combinations of the foregoing methods. The t-BOC and F-MOC methods, which are established and widely used, are described in Aarifield, J. Am. Chem. Soc., 88:2149 (1963); Meienhofer, Hormonal Proteins and Peptides, C. H. Li, Ed., Academic Press, 1983, pp. 48-267; and Barany and Aarifield, in The Peptides, E. Gross and J. Meienhofer, Eds., Academic Press, New York, 1980, pp.3-285. Methods of solid phase peptide synthesis are described in Aarifield, R. B., Science, 232:341 (1986); Carpino, L. A. and Han, G. Y., J. Org. Chem., 37:3404 (1972); and Gauspohl, H. et al., Synthesis, 5:315 (1992)). The teachings of these references are incorporated herein by reference.

As indicated above the compounds of the invention may be prepared utilizing various peptidic cyclizing techniques. Methods of cyclizing compounds having peptide sequences are described, for example, in Lobl et al., WO 92/00995, the teachings of which are incorporated herein by reference. Cyclized molecules can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or ornithine, for example) or acetamidomethyl (Acm) (for the sulfhydryl of cysteine) protecting groups. OAI and Aloc are easily removed by Pd and Acm is easily removed by iodine treatment.

Other modes of cyclization (beyond N- to C- terminal cyclization) may include: N- to backbone cyclization, C- to backbone cyclization, N- to side chain cyclization, C- to side chain cyclization, backbone to side chain cyclization, backbone to backbone cyclization and side chain to side chain cyclization.

8. Determination of Kinase-associated Signal Transduction Modulating Activity

It should be appreciated that some of the compounds that comprise sequences (b1)-(b6) above have modulating activities of the signal transduction associated with the kinase while some do not. Some of the conservative substitutions in the essential positions may diminish the modulating activities altogether, while other such conservative substitution in the essential positions may improve these modulating activities. The same is true also for deletions, substitutions (both conservative and non-conservative) in non-essential positions, as well as to chemical modifications (in any position) or insertions. In addition the type and size of the non-amino acid portion of the compounds, such as a hydrophobic moiety in one of its terminals may diminish or increase the modulation of the signal transduction. Those compounds which fall under the scope of the present invention are those that have signal transduction modulating activities, which activities that can be determined for example by using one of the assays stipulated below.

8.1 Cellular Assay

It can be readily determined whether a compound modulates the signal transduction associated with a kinase by incubating the compound with cells which have one or more cellular activities controlled by the signal transduction. Examples of these cellular activities include cell proliferation, cell differentiation, cell morphology, cell survival or apoptosis, cell response to external stimuli, gene expression, lipid metabolism, glycogen or glucose metabolism and mitosis. The cells are incubated with the candidate compound to produce a test mixture under conditions suitable for assessing the level of the signal transduction associated with the kinase. The activity of the signal transduction is assessed and compared with a suitable control, e.g., the activity of the same cells incubated under the same conditions in the absence of the candidate compound (or in the presence of a control compound). A greater or lesser activity of the signal transduction in the test mixture compared with the control indicates that the candidate compound modulated the signal transduction associated with the kinase.

Suitable cells for the assay include normal cells which express the membrane bound or intracellular kinases, cells which have been genetically engineered to express a kinase, malignant cells expressing a kinase or immortalized cells that express the kinase.

Conditions suitable for assessing activity include conditions suitable for assessing a cellular activity or function under control of the signal transduction associated with the kinase pathway. Generally, a cellular activity or function can be assessed when the cells are exposed to conditions suitable for cell growth, including a suitable temperature (for example, between about 30° C. to about 42° C.) and the presence of the suitable concentrations of nutrients in the medium (e.g., amino acids, vitamins, growth factors or of specific activators such as cytokines, hormones and the like).

For example, the level of kinase-associated signal transduction (e.g., Akt/PKB, Dudek et al., Science 275:661 (1997)) can be evaluated by growing the cells under serum deprivation conditions. Cells are typically grown in culture in the presence of a serum such as bovine serum, horse serum or fetal calf serum. Many cells, for example, nerve cells such as PC-12 cells, generally do not survive with insufficient serum. The use of insufficient serum to culture cells is referred to as “serum deprivation conditions” and includes, for example, from 0% to about 4% serum. Kinase-associated signal transduction is determined by the extent to which a peptide or peptide derivative can protect cells, e.g., neuronal cells, from the consequences of serum deprivation. Specific conditions are provided in Dudek et al., and in Example 4 of the application entitled “SHORT PEPTIDES WHICH SELECTIVELY MODULATE INTRACELLULAR SIGNALING” (filed on May 21, 1997, U.S. application Ser. No. 08/861,153), the pertinent teachings of which are incorporated herein by reference.

Generally, the level of the signal transduction associated with the kinase in the test mixture is assessed by making a quantitative measure of the cellular activity which the kinase-signaling controls. The cellular activity can be, for example, cell proliferation. Examples of cells in which proliferation is controlled by a kinase-associated signal transduction include endothelial cells such as bovine aortic cells, mouse MSI cells or mouse SVR cells (see Arbiser et al., Proc. Natl. Acad. Sci. USA 94:861 (1997)), vascular smooth muscle cells, fibroblasts of various tissue origin, and malignant cells of various tissues such as breast cancer, lung cancer, colon cancer, prostate cancer or melanoma. Signal transduction associated with the kinase is assessed by measuring cellular proliferation, for example, by comparing the number of cells present after a given period of time with the number of cells originally present. One example of kinases having to do with cellular proliferation are the receptors of the activin-like kinases (ALKs) super-family.

If cells are being used in which the kinase-associated signal transduction controls cell differentiation (e.g., PC-12 cells transfected with c-Src, see Alema et al., Nature 316:557 (1985)), the modulating activity is assessed by measuring the degree of differentiation. Activity can be assessed the degree to which neurites are extended and the degree to which markers of neuronal differentiation are expressed in PC-12 cells transfected with c-Src; see Alema et al., and the degree to which the formation of mesoderm in developing Xenopus embroya cells is induced; see Burgess and Maciag, Ann. Rev. Biochem, 58:575 (1989) and Dionne et al., WO 92/00999. Activity can also be assessed by the extent to which gene expression, cell morphology or cellular phenotype is altered (e.g., the degree to which cell shape is altered or the degree to which the cells assume a spindle-like structure). One example of a change in cellular morphology is reported in the application entitled “SHORT PEPTIDES WHICH SELECTIVELY MODULATE INTRACELLULAR SIGNALING” (filed on May 21, 1997, U.S. application Ser. No. 08/861,153), which discloses that certain peptide derivatives of the HJ loop of protein tyrosine kinases can cause vascular smooth muscle cells to become elongated and assume a spindle-like shape. A specific example of cellular assay is glucose uptake as specified in Example 2 bellow or lipogenesis by adipose cells.

8.2 Phosphorylation of substances

Where the substrates of the kinases are known, it is possible to assess the kinase-associated signal transduction and the changes in this signal as compared to control, by determining the phosphorylation level of the substrate protein. Cells known to express the kinase are incubated with a candidate compound for modulating the signal transduction. Then the cells are lysed, the protein content of the cells is obtained and separated on a SDS-PAGE. The substrates can be identified by use of suitable molecular weight markers, or by using suitable antibodies. The level of substrate phosphorylation can be determined by using anti-phosphotyrosine antibodies, either conjugated to a suitable label or further reacted with a label-bearing antibody (see Fujimoto et al., Immunity, 13:47-57 (2000)).

Alternatively phosphorylation may be determined in a cell-free system by incubating a mixture comprising kinases, the substrate of the kinase and candidate molecules for modulating kinase-associated signal transduction in the presence of ATP under conditions enabling phosphorylation. The proteins are then subjected to SDS-PAGE, transferred to nitrocellulose (where the substrate band is identified by antibody or molecular weight marker followed by immunoblotting by anti-phosphotyrosine antibody. Alternatively it is possible to use [γ-³² P] ATP and quantify the amount of radioactivity in cooperated in the substrate (See Fujimoto et al., The J. of Immunol. 7088-7094 (1999).

It should be appreciated that the specific assay should be designed in accordance with the activities of the specific kinase to be modulated by the compound.

8.3. Tissue or in vivo Assay

Suitable assays for determining modulation of kinase-associated signal transduction can also be prepared, according to the specific tissue.

An example is modulation of IRK-associated signal transduction by measuring the level of glucose in an animal model of Diabetes for example as described below in Example 3. Another example is modulation of neurite extension as shown in Example 4.

9. Using the Compounds of the Invention to Identify Ligands

The A-region within kinase plays a key role in the kinase associated signal transduction. The compound comprising the A-region peptides of the present invention can also be used to identify ligands which interact with the A-regions of a specific kinase and thus can modulate the kinases-associated signal transduction. For example, an affinity column can be prepared to which a specific A-region peptide is covalently attached, directly or via a linker. This column, in turn, can be utilized for the isolation and identification of specific ligands which bind the A region peptide and which will also likely bind the kinase from which the A-region peptide was derived. The ligand can then be eluted from the column, characterized and tested for its ability to modulate kinase function. The peptides may also be used as a research tool for identifying the components with which the kinase interacts.

### EXAMPLE 1

### Preparation of Compounds of the Invention

The novel compounds of this invention can be synthesized utilizing a 430A Peptide Synthesizer from Applied Biosystems using F-Moc technology according to manufacturer's protocols. Other suitable methodologies for preparing sequences are known to person skilled in the art. See e.g., Aarifield, R. B., Science, 232: 341 (1986); Carpino, L. A., Han, G. Y., J. Org. Chem., 37: 3404 (1972); Gauspohl, H., et al., Synthesis, 5: 315 (1992)), the teachings of which are incorporated herein by reference.

Rink Amide Resin [4(2′,4′Dimethoxyphenyl-FMOC amino methyl) phenoxy resin] was used for the synthesis of C-amidated peptides. The alpha-amino group of the amino acid was protected by an FMOC group, which was removed at the beginning of each cycle by a weak base, 20% piperidine in N-methylpyrrolidone (NMP). After deprotection, the resin was washed with NMP to remove the piperidine. In situ activation of the amino acid derivative was performed by the FASTMOC Chemistry using HBTU (2(1-benzotriazolyl-1-yl)-1,1,3,3-tetramethyluronium) dissolved in HOBt (1-hydroxybenzotriazole) and DMF (dimethylformamide). The amino acid was dissolved in this solution with additional NMP. DIEA (diisopropylethylamine) was added to initiate activation. Alternatively, the activation method of DCC (dicyclohexylcarbodiimide) and HOBt was utilized to form an HOBt active ester. Coupling was performed in NMP. Following acetylation of the N-terminus (optional), TFA (trifluoroacetic acid) cleavage procedure of the peptide from the resin and the side chain protecting groups was applied using 0.75 g crystalline phenol; 0.25 ml EDT (1,2-ethandithiol); 0.5 ml thioanisole; 0.5 ml D.I. H₂O; 10 ml TFA.

### EXAMPLE 2

### Glucose Uptake

Experimental Procedures

A. Materials and Solutions

Materials

30 ml plastic bottle (Nalgene 2103-0001)

50 ml plastic conical tube (Miniplast 204-21)

2 ml microcentrifuge tube

0.4 ml microtubes (Sarstedt 72.7000)

250 μ nylon mesh

Collagenase Type 1 (Worthington, type I, CLS 4196)

Dinonyl phthalate (Aack 1.09669.0100)

3 H-Deoxy Glucose (Net 549A), 29.8 Ci/mmole, 0.25 mCi, 0.25 ml

Scintillation tubes (ultraplast, 3951)

2-3 male rats, Sprague Dawley, 150-200 gr each

Solutions

Krebs Ringer Bicarbonate HEPES buffer (KRBH), containing 1% bovine fraction 5 albumin and 200 nM adenosine was made, using stock solutions:

Stock solution 1—salts

| | |
|---|---|
| 1.2 M | NaCl |
| 40 mM | KH2PO4 |
| 10 mM | MgSO4 |
| 10 mM | CaCl2 (Dissolved |
| | in a small flask |
| | and added to |
| | other salts). |

Stock solution 2—Sodium bicarbonate

100 mM NaHCO3

Stock solution 3 - HEPES

| | |
|---|---|
| 30 mM | HEPES pH 7.4 at 37° C. |

15 ml of each stock solution (1, 2 and 3) and 15 ul of stock solution 4 were added to 105 ml double distilled water on day of use. 1.5 gr BSA fraction V were then added.

B. Adipose Cell Isolation Procedure

To 3 ml of KRBH buffer with 10 mg collagenase, 3g epididymal fat pad (from 2-3 male rats lightly ether anesthesised and then decapitated) was introduced. The fat was cut up with scissors. The pieces of fat were swirled and shaken in the collagenase solution in a 37° C. water bath, set at 100-150 repetitions/minute, for approximately 1 hour with swirling every 15 minutes while digesting and every 5 minutes towards the end. About 6 ml of buffer was then added to the vial. The content of the vial was gently squeezed through a 250 μ nylon mesh into a 50 ml plastic tube. The residual fat tissue was washed (the total volume for each wash was 15 ml): the tube was centrifuged whereby the adipose cells floated at the top of the liquid. The buffer was removed using a syringe with polyethylene capillary. Buffer was added to get 15 ml and clumps of adipose cells were broken up by gently mixing up and down. This procedure was repeated for a total of 4 centrifugations: 3 centrifugations at 1000 rpm with the last centrifugation at 2000 rpm. At this point, the buffer was removed. Fresh buffer was added to the cell suspension to form a cytocrit of 5-10%. The cells were rolled at 10 rpm at 37° C. for 1 hour.

C. Glucose Uptake Procedure

50 μl 0.1% BSA in PBS was placed with compounds comprising IRK-derived peptides (final concentrations: 0.1-50 μM) or the peptide-vehicle in each 2 ml plastic tube in duplicates. 950 μl aliquots of the cell suspension were added to the tubes. After incubation for 1 hour at 37° C. in a roller (10 rpm), 200 ul of insulin was added to certain tubes to get final concentrations of 2.5-1000 uU/ml and than 200 μl of KRBH buffer containing 3H—Deoxy Glucose (approx. 1200 dpm/μl) was added to each tube. After 30 minutes incubation with the 3H-DOG and insulin at 37° C., 200 μl aliquots were transferred to microcentrifuge tubes containing 200 μl Dinonyl phthalate in duplicates. Cells were rapidly separated from the aqueous buffer by centrifugation at 10,000 g for 60 sec. The cells were isolated in the top layer of the dinonyl phthalate phase. The upper part of the tube (which contains the cells) was cut and transferred to scintillation tube. 4 ml of scintillation liquid was added.

Cell associated radioactivity was counted in a liquid scintillation beta counter.

D. Increase of Glucose-Uptake by Compounds Comprising IRK-Derived Sequences

Glucose-uptake was measured in fresh adipocytes, incubated with or without insulin (10 μ/ml) as described above, in the absence (vehicle) or the presence of 10 μM of a compound K094A107 (“107”) SEQ ID NO: 102; or K094A205 (“205”) (SEQ ID NO: 123), which was kept in a reducing environment (5 to 25 μM DTT) As a comparison the glucose uptake with insulin alone was determined. The results of this uptake are shown in FIGS. 5A and 5B.

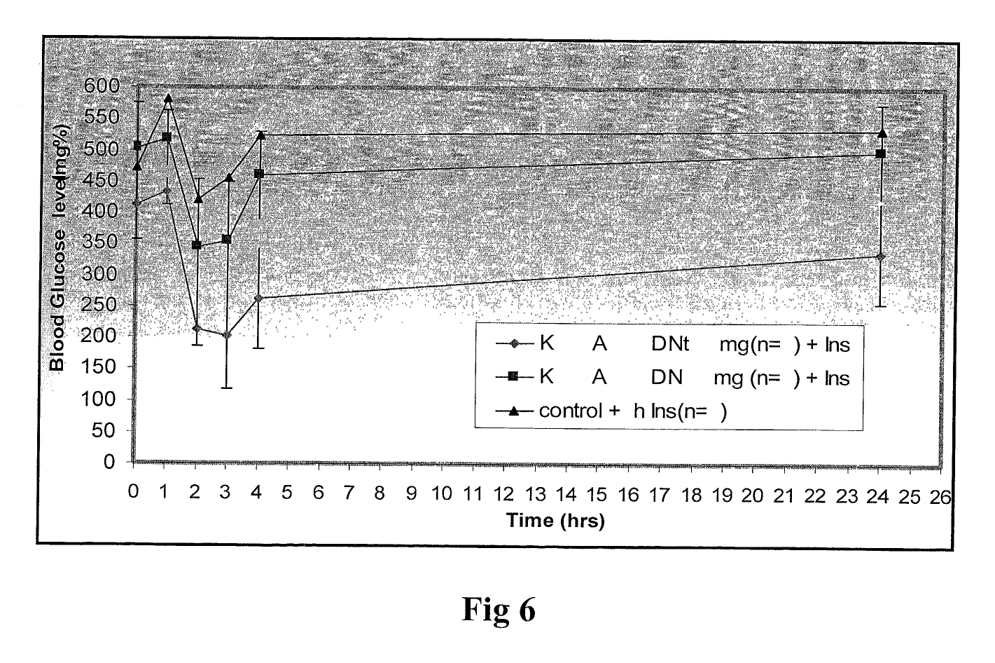

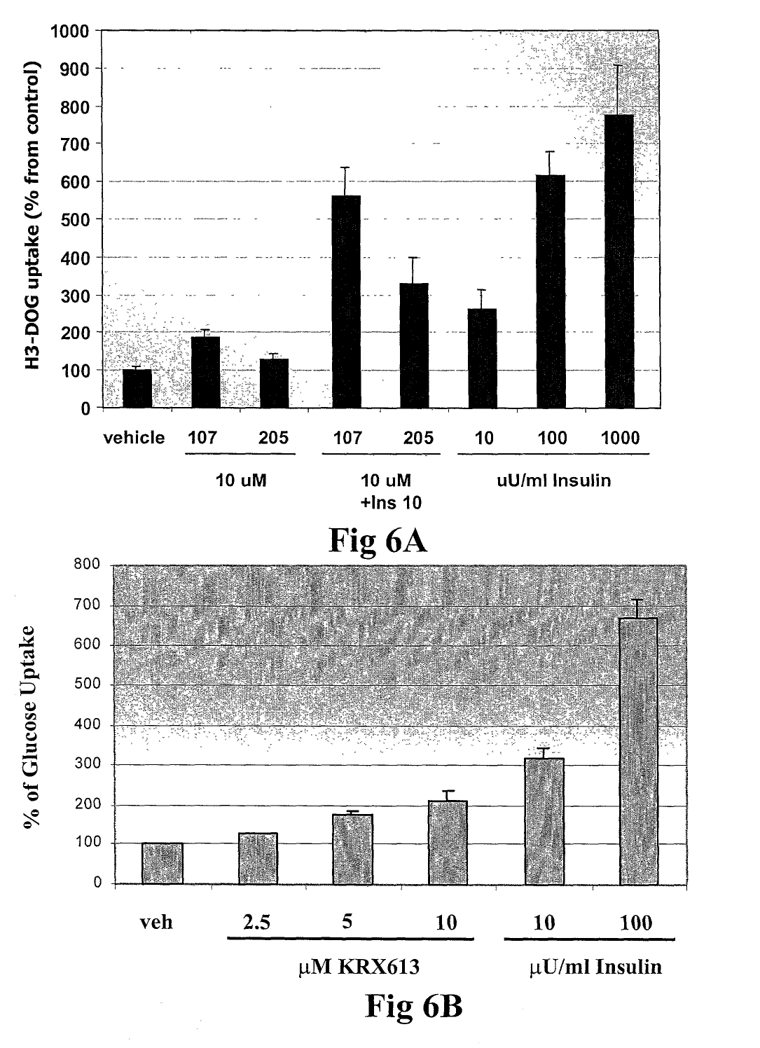

As can be seen in FIG. 6A, both “107” and “205” were able to increase glucose uptake levels beyond the level of control glucose uptake. The effect of “107” in the presence of 10 μU/ml insulin was ever more striking as it was an synergistic. FIG. 6B shows that “107” was able to induce glucose uptake in a dose-dependent manner.

### Example3

### In Vitro Decrease of Blood Glucose by the Compound of the Invention

Diabetes was induced in male Sabra rats (150-200 gr) by I.P. injection of streptozotocin (Sigma, S0130) 60 mg/Kg dissolved in ph 4.5 saline (Biochemical and biophysical research comunications 197(3): 1549-1555 (1993)), which is known to destroy insulin-secreting pancreatic cells. After 2-7 days the rats were all diabetic, as determined by blood glucose level above 200 mg/dl which was measured with glucometer.

12 rats were divided into 3 groups, with homogenic blood glucose level, each consisting of 4 animals

Group I was administered with K094A107 (SEQ ID NO: 102) 10 mg/animal (I.P.) followed by administration of insulin 0.125 units/animal (low concentration of insulin that nearly doesn't give any significant decrease in blood glucose level) one hour later.

Group II was administered with K094A205 (A-region in SEQ ID NO: 123) 10 mg/animal (I.P.) followed by insulin 0.125 units/animal one hour later.

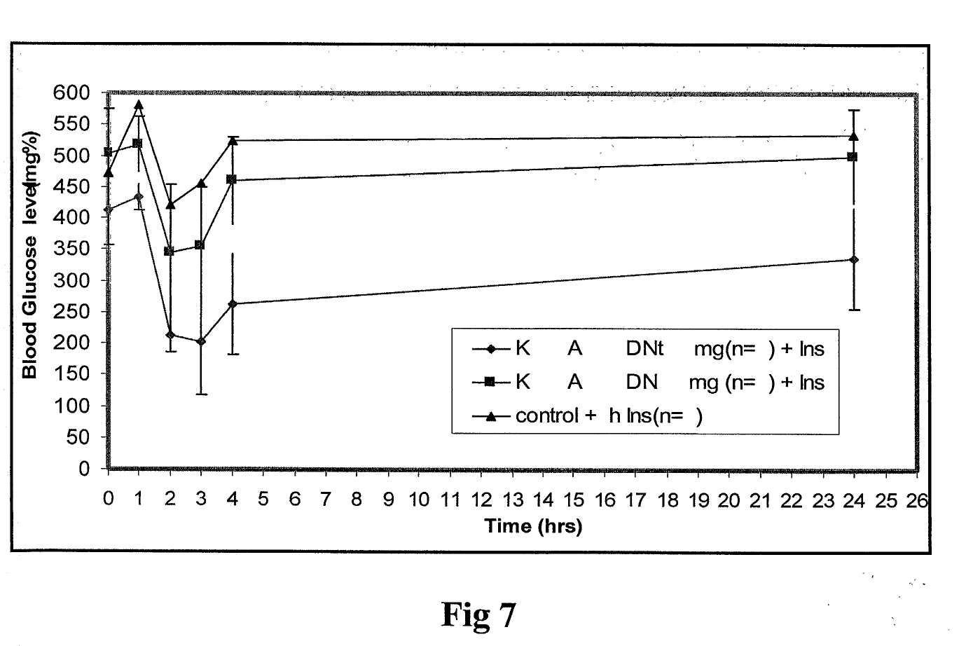

Group III served as control and was administered I.P. with vehicle (NAC/DMSO-DIDV) followed by insulin 0.125 units/animal one hour later. Glucose levels in blood were measured for 24 hours by Glucometer and the results are shown in FIG. 7.

As can be seen, 205 caused a marked reduction in glucose blood levels in all periods tested, and the effect of a single administration were notable even 24 hours later.

### EXAMPLE 4

### Enhancement of Emigration of Neural Crest Cells from Neural Primordia by the Compound of the Invention

The onset of neural crest cell migration is a complex morphogenetic process. A balance between BMP-4 and its inhibitor noggin regulate emigration of neural crest progenitors from the neuroepithelium.

The procedure for explants of neural primordia is described in greater detail in Sela-Donefeld and Kalcheim (Development, 125(21): 4749-4762 (1999)), the teaching of which was incorporated herein by reference.

The trunk region of 16 somite-old quail embryos was separately sectioned at the level of the segmental plate plus the last 2 epithelial somite pairs. Neural primordia consisting of the neural tube and premigratory neural crest cells were isolated from adjacent tissues with 25% pancreatin in PBS, transferred to PBS supplemented with 5% newborn calf serum to stop enzymatic activity and washed in serum-free culture medium prior to explanation. The neural primordia were then explanted onto multi-well chamber slides that were pre-coated with fibronectin (50 μg/ml) for 1 hour. The neural primordia were cultured in 50 μl of either serum-free CHO—S-SFMII medium (GibcoBRL, USA) or condition medium of noggin producing-CHO cells, in the absence or presence of the tested compound, in a final concentration of 5 μM and incubated in a humid chamber for 24 hours. At the end of the incubation, the primordia were gently washed with PBS and fixed with Bouin's fluid, washed 3 times with PBS, dried and covered.

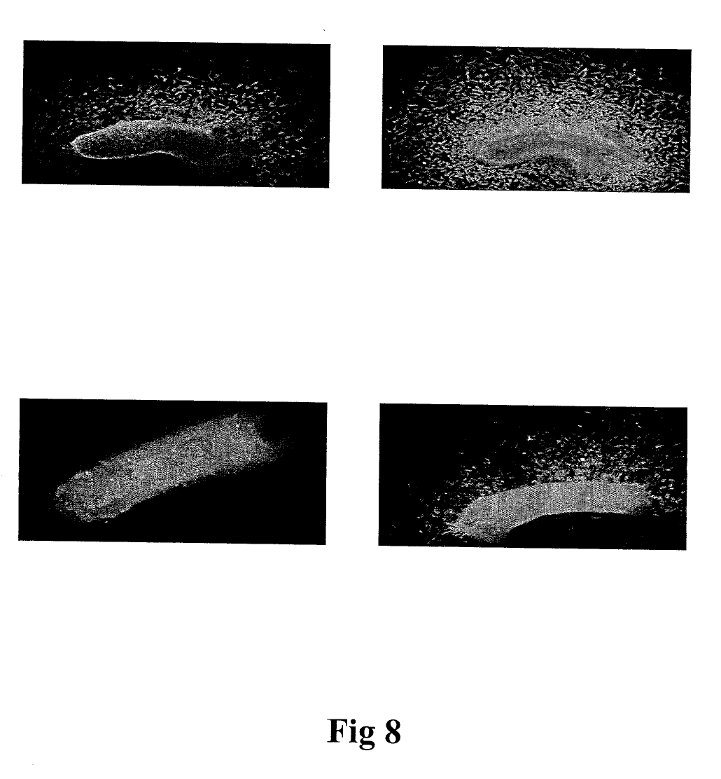

The results for a compound comprising ALK-3-derived peptide (K098A100 SEQ ID NO: 115) are shown in FIG. 8.

As can be seen in FIG. 8 (top-left two pictures), neural crest cells naturally migrate out of the tube. In the presence of the compound (FIG. 7 top-right two pictures), the number of migrating cells is higher indicating that the compound was capable of mimicking BMP effects of the neural cells. Noggin, which is a specific inhibitor of BMP-4, blocks the neural crest migration (FIG. 7 bottom-left two pictures). However, in the presence of noggin and compound (FIG. 7 bottom-right two pictures), the compound overcomes this inhibition and induces neural crest migration (albeit at a lower level than in the absence of noggin).

These results show that a compound of the invention comprising an ALK-3 derived sequence can modulate a signal transduction controlled over neural crest migration.

### EXAMPLE 5

### ALA-SCAN

Glucose uptake as described in Example 2 was tested with a plurality of compounds of the invention (SEQ ID NO: 124-133) all derived from IRK, wherein in each sequence sequentially one amino acid was replaced by Alanine. For some sequences the procedure was tested twice. As can be seen, when most amino acids were replaced by Ala, the glucose uptake was not significantly altered indicating that most amino acids are not essential for the signal transduction associated activity,. Those amino acids found essential (i.e. their replacement cause adecrease in glucose uptake as compared to the parent unmodified sequence) Gly, Val, Arg should be maintained, conservatively substituted or chemically modified.

### EXAMPLE 6

### Change in Phosphorylation of Substrates

All experiments were carried out with H4 cells (Rat hepatoma cell line).Cells were pre-incubated with the compound K094A107 comprising an IRK-derived peptide (SEQ ID NO: 102)(10mM) or the corresponding controls for 3 hours ( one control was control -no treatment vehicle only, the second control was an irrelevant peptide derived from a non-A-region of a kinase), and with human recombinant insulin (100 nM), for 5 minutes. The cells were then lysed and cell lysates were subjected to Western Blot analysis followed by detection with ECL.

The membrane was incubated overnight with a primary polyclonal Anti-PhosphoPKB (phospho-serine 473 PKB)Ab, or with an antibody for IRK and then, after 3 washes with TBST, with secondary HRP antibody was added for 1 hour. In the second gel the membrane was incubated overnight with a primary monoclonal PhosphoTyrosine Kinase (PY99) Ab that labels phosphorylated tyrosine, and then after 3 washes with TBST, a secondary antibody was added for 1 hour.

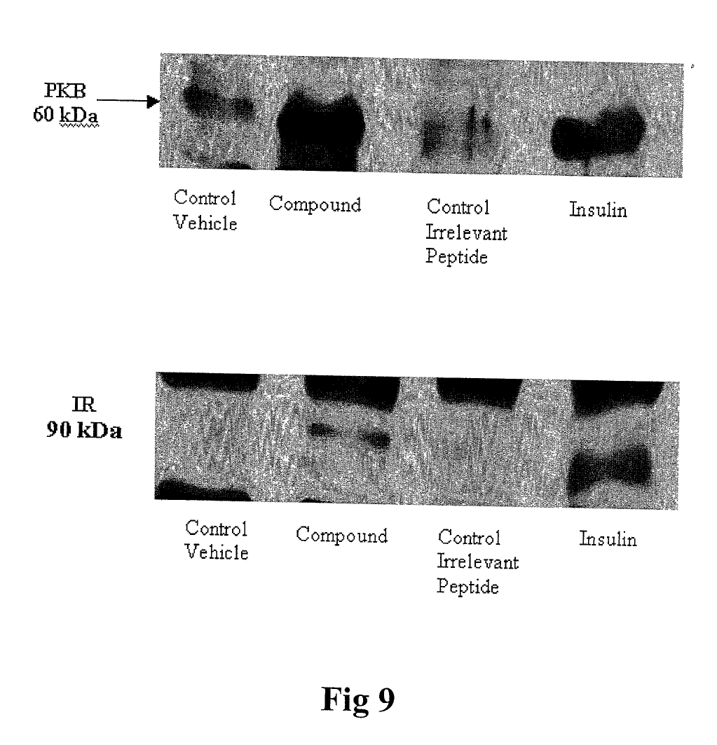

The results are shown in FIG. 9. As can be seen a compound comprising an A-region derived IRK- peptide was able to increase phosphorylation of substrates that are two kinases in the IRK-signal transduction pathway, IRK (which auto-phosphorylates) and PKB, in a manner similar to phosphorylation induced by insulin, thus modulating the kinase-associated signal transduction . This modulation can result for example in an increase in glucose uptake and decrease of blood glucose as shown in examples 2 and 3 above. Another peptide derived from a different, non A-region of another kinase (GRK) was unable to cause said change indicating that the effect on the IRK signaling pathway is by peptides derived from the IRK kinase.

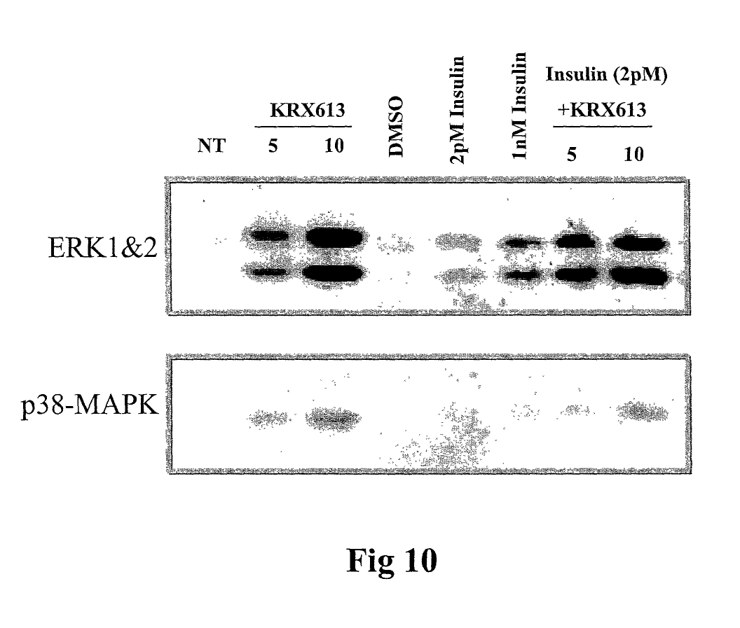

A similar experiment was conducted with the difference being that the substrates tested were the substrates of the kinase associated signal transduction that are more downstream in the pathway: Erk1, and p38-MAP. The results are shown in FIG. 10. As can be seen the compound of SEQ ID NO: 102 (“613”) was able to increase the phosphorylation of ERK1 and p38-MAP in a dose dependent manner, to levels which were higher than phosphorylation caused by insulin indicating that the modulation of signal transduction can be determined not only by the determination of the level of phosphorylation of the direct substrate of the kinase (from which the A-region was derived) but also by the determination of the level of substrates which are more down stream in the signal transduction pathway.

1. A method for identifying candidate compounds for the modulation of kinase-associated signal transduction the method comprising:
(a) identifying a peptide region in the kinase (“A-region”) by aligning catalytic subunits of the kinase and PKA-Cα and determining the sequence of the kinase corresponding to positions 92-109 of PKA-Cα;
(b) synthesizing at least one compound comprising a sequence selected from the group consisting of:
(b1) a sequence comprising of from a minimum of 5 continuous amino acids of said A-region to a maximum of all the continuous amino acids of said A-region;
(b2) a variant of the sequence of (b1) wherein up to 40% of the amino acids of the sequence of (b1) have been replaced with a naturally or non-naturally occurring amino acid or with a peptidomimetic organic moiety; and/or up to 40% of the amino acids have their side chains chemically modified,; and/or up to 20% of the amino acids have been deleted; provided that at least 50% of the amino acids of (b1) are maintained unaltered in the variant;
(b3) a sequence of (b1) or (b2) wherein one or more of the amino acids is replaced by the corresponding D-amino acid;
(b4) a sequence of any one of (b1) to (b3) wherein at least one peptidic backbone atom, or peptidic backbone bond has been altered to a modified peptidic backbone atom or a non-naturally occurring peptidic backbone bond, respectively;
(b5) a sequence of any one of (b1), (b2), (b3) or (b4) in a reverse order; and
(b6) a combination of two or more of the sequences of (b1), (b2), (b3), (b4) or (b5); and
(c) testing each compound of (b) to determine the capacity thereof to modulate the signal transduction associated with the kinase.

2. A method according to claim 1, wherein the determination of the signal transduction associated with the kinase is by determination of the level of phosphorylation of at least one kinase-substrate, and wherein step (c) comprises subjecting cellular components of the signal transduction to the presence or absence of the compound, and exterminating whether the presence of said compound caused change in the level of phosphorylation of the least one substrate as compared to the level of phosphorylation in the absence of the compound.

3. A method according to claim 1, further including after step (a) and prior to the step(b) the following steps
(a.i) determining a continuous stretch of at least 5 amino acids of the A-region identified in claim 1(a) above, that is shorter than the length of the full A-region and modulates the signal transduction associated with the kinase, by synthesizing a plurality of compounds each comprising one of a plurality of subsequences (optionally partially overlapping subsequences) of 5-10 aa which are present as a continuous sequence in the A-region; testing those compounds in a test assay for determining signal transduction associated with the kinase, and selecting those subsequences that modulates said kinase-associated signal transduction; and
(a.ii) determining in the sequences of (a.i) essential and non-essential amino acids by: preparing a plurality of modified sequences wherein in each sequence a single and different amino acid of the native sequence has been replaced with a test amino acid to produce modified sequences; testing those modified sequences in a test assay for determining signal transduction associated signal transduction, identifying as essential amino acids those amino acids which when replaced, caused a statistically significant change in signal transduction;
and wherein said sequence of (b1) is a sequence determined by step(a.i) and wherein said sequence of (b2) is the sequence, wherein at least one of the essential amino acids identified by the step of (a.ii) has been replaced by a conservatively substituted naturally or non-naturally occurring amino acid, or a conservative peptidomimetic organic moiety, and/or wherein at least one of the non-essential amino acids has been deleted, or substituted (conservatively or non-conservatively) by naturally or non-naturally occurring amino acids or a peptidomimetic organic moiety.

4. A method according to claim 3 wherein the test amino acid is Alanine.

5. A method for obtaining a compound for the modulation of kinase associated signal transduction the method comprising:
(a) identifying candidates for the modulation of signal transduction associated with the kinase according to the method of claim 1;
(b) selecting from the candidates of (a) a compound that modulates signal transduction associated with the kinase in the test assay as compared to the modulation of the signal transduction associated with the kinase in the same test assay in the absence of the compound; and
(c) producing the compound of (b) thereby, obtaining compounds for the signal transduction associated with the kinase.

6. A method according to claim 5, wherein the test assay for determining kinase-associated signal transduction is selected from the group consisting of:
(a) an assay wherein the level of phosphorylaton of at least one substrate of the kinase is determined;
(b) an assay wherein the level of at least one of the following kinase-associated signal transduction-dependent cellular properties is determined: proliferation, differentiation, cellular-shape alteration, cellular elongation, glucose uptake by cells, lipogenesis by adipose cells, and secretion of substances from cells; and
(c) an in vivo assay wherein the level of at least one of the following kinase-associated signal transduction physiological properties is determined: level of metabolites, hormones ,or cytokines in circulation; size of induced or implanted tumor, number of metastases; weight alteration; appetite alteration, infection level; inflammation level; level of tissue remodeling including bone healing, scar formation, fibrous deposition, alopecia, adipose formation; level of neurite extension; glucose levels in blood, .

7. A compound for the modulation of signal transduction associated with a kinase obtained by the method of claim 5.

8. A compound which has the property of modulation of signal transduction of a kinase consisting of: at least one moiety for transport across cellular membranes, in association with a sequence selected from the group consisting of:
(1) a sequence comprising of from a minimum of 5 continuous amino acids of said A-region to a maximum of all the continuous amino acids of said A-region;
(2) a variant of the sequence of (1) wherein up to 40% of the amino acids of the sequence of (1) have been replaced with a naturally or non-naturally occurring amino acid or with a peptidomimetic organic moiety; and/or up to 40% of the amino acids have their side chains chemically modified, and/or up to 20% of the amino acids have been deleted, provided that at least 50% of the amino acids of (1) are maintained unaltered in the variant;
(3) a sequence of (1) or (2) wherein one or more of the amino acids is replaced by the corresponding D-amino acid;
(4) a sequence of any one of (1) to (3) wherein at least one peptidic backbone atom, or peptidic backbone bond has been altered to a modified peptidic backbone atom or a non-naturally occurring peptidic backbone bond, respectively;
(5) a sequence of any one of (1), (2), (3) or (4) in a reverse order; and
(6) a combination of two or more of the sequences of (1), (2), (3), (4) or (5).

9. A compound according to claim 8, wherein the moiety is a hydrophobic moiety.

10. A method for the modulation of a signal transduction associated with a kinase, comprising contacting the kinase with a compound according to claim 7 or 8.

11. A method for the modulation of signal transduction associated with a kinase in a subject comprising administering to the subject a therapeutically effective amount of a compound according to claim 7 or 8.

12. A method for the treatment of a disease, wherein a therapeutically beneficial effect may be evident by the modulation of a signal transduction associated with a kinase, the method comprising: administering to a subject in need of such treatment a therapeutically effective amount of a compound according to claim 7 or 8, wherein the kinase from which the A-region is determined, is the kinase associated with said signal transduction

13. A method according to claim 12, for the treatment of a disease selected from: diabetes, cancer, obesity, restenosis, tissue remodeling including: improved bone healing, prevention of alopecia, reduced scarring, osteoporosis, neurodegenerative disease, autoimmune disease, inflammation, restenosis an, atherosclerosis, skin disorders, diseases of the central nervous system, inflammatory disorders, autoimmune diseases and other immune disorders, osteoporosis and cardiovascular diseases.

14. A compound according to claim 7 or 8, comprising a sequence corresponding to at least 5 continuous amino acids present in a sequences selected from any one of SEQ ID NOS: 54 to 133.

15. A compound according to claim 7 or 8, comprising a variant of a sequence that corresponds to at least 5 continuous amino acids of any one of the sequences of SEQ ID NOS: 54 to 133, the variant obtained by replacing up to 40% of the amino acids with a naturally occurring, non-naturally occurring or peptidomimetic organic moiety, and/or chemically substituting up to 40% of the amino acids' side chains, and/or deletion of up to 20% of the amino acids, provided that at least 50% of the amino acids of any one of SEQ ID NO: 54 to 133 are maintained unaltered in the variant.

16. A method according to claim 13, wherein the disease is diabetes and the kinase is IRK.

17. A method according to claim 16, wherein the compound is selected from K094A107 (SEQ ID NO: 102) and K094A205 (SEQ ID NO: 123).

18. The method of claim 1, wherein the kinase is selected from the group consisting of SRC, LYN, HCK and LCK protein and the sequence of the A-region is selected from the group consisting of SEQ ID NO: 1, SEQ ID NO: 2, SEQ ID NO: 3, SEQ ID NO: 4, SEQ ID NO: 5, SEQ ID NO: 6 and SEQ ID NO: 7, respectively.

19. A method according to claim 18, wherein the sequence (b1), (b2) or (b3) consists of five to eighteen amino acids present in AA₁ to AA₁₈ wherein:
AA₁ is selected from the group consisting of alanine and glycine;
AA₂ is selected from the group consisting of glutamine, asparagine, glutamic acid, aspartic acid and an aliphatic, substituted aliphatic, benzyl, substituted benzyl, aromatic or substituted aromatic ester of a glutamic or aspartic acid;
AA₃ is selected from the group consisting of valine, isoleucine, leucine and methionine;
AA₄ is selected from the group consisting of methionine, isoleucine, leucine and valine;
AA₅ is selected from the group consisting of lysine and arginine;
AA₆ is selected from the group consisting of lysine, leucine, threonine, glutamine, arginine, isoleucine, methionine, valine, serine, glutamic acid, asparagine, aspartic acid and an aliphatic, substituted aliphatic, benzyl, substituted benzyl, aromatic or substituted aromatic ester of a glutamic or aspartic acid;
AA₇ is selected from the group consisting of leucine, isoleucine, methionine, and valine;
AA₈ is selected from the group consisting of arginine, lysine, glutamine, glutamic acid, aspartic acid, asparagine and an aliphatic, substituted aliphatic, benzyl, substituted benzyl, aromatic or substituted aromatic ester of a glutamic or aspartic acid;
AA₉ is histidine;
AA₁₀is selected from the group consisting of glutamic acid, aspartic acid, glutamine, asparagine and an aliphatic, substituted aliphatic, benzyl, substituted benzyl, aromatic or substituted aromatic ester of a glutamic or aspartic acid;
AA₁₁ is selected from the group consisting of lysine and arginine;
AA₁₂ is selected from the group consisting of leucine, isoleucine, methionine and valine;
AA₁₃ is selected from the group consisting of valine, isoleucine, leucine and methionine;
AA₁₄ is selected from the group consisting of glutamine, proline, arginine, lysine, glutamic acid, aspartic acid, asparagine and an aliphatic, substituted aliphatic, benzyl, substituted benzyl, aromatic or substituted aromatic ester of a glutamic or aspartic acid;
AA₁₅ is selected from the group consisting of leucine, isoleucine, methionine and valine;
AA₁₆ is selected from the group consisting of tyrosine, histidine, phenylalanine and tryptophan;
AA₁₇ is selected from the group consisting of alanine and glycine; and
AA₁₈ is selected from the group consisting of valine, isoleucine, leucine and methionine.

20. A method according to claim 1, wherein the kinase is a CSK protein kinase and the sequence of the A-region is selected from the group consisting of by SEQ ID NO: 8 and SEQ ID NO: 9.

21. A method according to claim 20, wherein the sequence (b1), (b2) or (b3) consists of five to eighteen amino acids present in AA₁ to AA₁₈ wherein:
AA₁ is selected from the group consisting of alanine, threonine, glycine and serine;
AA₂ is selected from the group consisting of serine, alanine, threonine and glycine;
AA₃ is selected from the group consisting of valine, isoleucine, leucine and methionine;
AA₄ is selected from the group consisting of methionine, isoleucine, leucine and valine;
AA₅ is selected from the group consisting of threonine and serine;
AA₆ is selected from the group consisting of glutamine, lysine, glutamic acid, aspartic acid, asparagine, arginine and an aliphatic, substituted aliphatic, benzyl, substituted benzyl, aromatic or substituted aromatic ester of a glutamic or aspartic acid;
AA₇ is selected from the group consisting of leucine, methionine, isoleucine and valine;
AA₈ is selected from the group consisting of arginine, glutamine, lysine, glutamic acid, aspartic acid, asparagine and an aliphatic, substituted aliphatic, benzyl, substituted benzyl, aromatic or substituted aromatic ester of a glutamic or aspartic acid;
AA₉ is histidine;
AA₁₀ is selected from the group consisting of serine, glutamic acid, threonine, aspartic acid, glutamine, asparagine and an aliphatic, substituted aliphatic, benzyl, substituted benzyl, aromatic or substituted aromatic ester of a glutamic or aspartic acid;
AA₁₁ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₂ is selected from the group consisting of leucine, isoleucine, methionine and valine;
AA₁₃ is selected from the group consisting of valine, isoleucine, leucine and methionine;
AA₁₄ is selected from the group consisting of glutamine, arginine, glutamic acid, aspartic acid, asparagine, lysine and an aliphatic, substituted aliphatic, benzyl, substituted benzyl, aromatic or substituted aromatic ester of a glutamic or aspartic acid;
AA₁₅ is selected from the group consisting of leucine, isoleucine, methionine and valine;
AA₁₆ is selected from the group consisting of leucine, isoleucine, methionine and valine;
AA₁₇ is selected from the group consisting of glycine and alanine; and
AA₁₈ is selected from the group consisting of valine, isoleucine, leucine and methionine.

22. The method according to claim 1, wherein the kinase is an endothelial protein kinase and the sequence of the A-region is represented by SEQ ID NO: 12.

23. The method according to claim 22, wherein the sequence (b1), (b2) or (b3) consists of five to eighteen amino acids present in AA₁ to AA₁₈ wherein:
AA₁ is selected from the group consisting of leucine, isoleucine, methionine and valine;
AA₂ is selected from the group consisting of glutamic acid, aspartic acid, glutamine, asparagine and an aliphatic, substituted aliphatic, benzyl, substituted benzyl, aromatic or substituted aromatic ester of a glutamic or aspartic acid;
AA₃ is selected from the group consisting of valine, isoleucine, leucine and methionine;
AA₄ is selected from the group consisting of leucine, isoleucine, methionine and valine;
AA₅ is selected from the group consisting of cysteine and serine;
AA₆ is selected from the group consisting of lysine and arginine;
AA₇ is selected from the group consisting of leucine, isoleucine, methionine and valine;
AA₈ is selected from the group consisting of glycine and alanine;
AA₉ is histidine;
AA₁₀is histidine;
AA₁₁ is proline;
AA₁₂ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₃ is selected from the group consisting of isoleucine, leucine, methionine and valine;
AA₁₄ is selected from the group consisting of isoleucine, leucine, methionine and valine;
AA₁₅ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₆ is selected from the group consisting of leucine, isoleucine, methionine and valine;
AA₁₇ is selected from the group consisting of leucine, isoleucine, methionine and valine;
AA₁₈ is selected from the group consisting of glycine and alanine; and
AA₁₉ is selected from the group consisting of alanine and glycine.

24. A method according to claim 1, wherein the kinase is SRC, LYN, HCK or LCK protein and the sequence of the A-region is selected from the group consisting of SEQ ID NO: 13 and SEQ ID NO: 14.

25. A method according to claim 24, wherein the sequence (b1), (b2) or (b3) consists of five to eighteen amino acids present in AA₁ through AA_{19,} wherein:
AA₁ is selected from the group consisting of methionine, isoleucine, leucine, and valine;
AA₂ is selected from the group consisting of glutamic acid, glutamine, aspartic acid, asparagine and an aliphatic, substituted aliphatic, benzyl, substituted benzyl, aromatic or substituted aromatic ester of a glutamic or aspartic acid;
AA₃ is selected from the group consisting of valine, isoleucine, leucine and methionine;
AA₄ is selected from the group consisting of methionine, leucine, isoleucine and valine;
AA₅ is selected from the group consisting of lysine and arginine;
AA₆ is selected from the group consisting of methionine, leucine, isoleucine and valine;
AA₇ is selected from the group consisting of isoleucine, leucine, methionine and valine;
AA₈ is selected from the group consisting of glycine and alanine;
AA₉ is selected from the group consisting of lysine and arginine;
AA₁₀ is histidine;
AA₁₁ is selected from the group consisting of lysine and arginine;
AA₁₂ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₃ is selected from the group consisting of isoleucine, leucine, methionine and valine;
AA₁₄ is selected from the group consisting of isoleucine, leucine, methionine and valine;
AA₁₅ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₆ is selected from the group consisting of leucine, isoleucine, methionine and valine;
AA₁₇ is selected from the group consisting of leucine, isoleucine, methionine and valine;
AA₁₈ is selected from the group consisting of glycine and alanine; and
AA₁₉ is selected from the group consisting of alanine and glycine.

26. A method according to claim 1, wherein the kinase is selected from SRC, LYN, HCK or LCK protein and the sequence of the A-region is selected from the group consisting of SEQ ID NO: 15, SEQ ID NO: 16, SEQ ID NO: 17, SEQ ID NO: 18 and SEQ ID NO: 19.

27. A method according to claim 26, wherein the sequence (b1), (b2) or (b3) consists of five to eighteen amino acids present in AA₁ through AA₁₉ wherein:
AA₁ through AA₁₉ or a subsequence thereof comprising at least five amino acids, wherein:
AA₁ is selected from the group consisting of leucine, isoleucine, methionine and valine;
AA₂ is selected from the group consisting of lysine and arginine;
AA₃ is selected from the group consisting of isoleucine, leucine, methionine and valine;
AA₄ is selected from the group consisting of methionine, leucine, isoleucine and valine;
AA₅ is selected from the group consisting of threonine, serine, isoleucine, leucine, methionine and valine;
AA₆ is histidine;
AA₇ is selected from the group consisting of leucine, isoleucine, methionine and valine;
AA₈ is selected from the group consisting of glycine and alanine;
AA₉ is selected from the group consisting of proline, histidine, asparagine, glutamine, glutamic acid, aspartic acid and an aliphatic, substituted aliphatic, benzyl, substituted benzyl, aromatic or substituted aromatic ester of a glutamic or aspartic acid;
AA₁₀ is histidine;
AA₁₁ is selected from the group consisting of leucine, isoleucine, methionine an valine;
AA₁₂ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₃ is selected from the group consisting of isoleucine, valine, leucine and methionine;
AA₁₄ is selected from the group consisting of valine, isoleucine, leucine and methionine;
AA₁₅ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₆ is selected from the group consisting of leucine, isoleucine, methionine and valine;
AA₁₇ is selected from the group consisting of leucine, isoleucine, methionine and valine;
AA₁₈ is selected from the group consisting of glycine and alanine; and
AA₁₉ is selected from the group consisting of alanine and glycine.

28. A method according to claim 1, wherein the kinase is selected from the group consisting of SRC, LYN, HCK and LCK protein and the sequence of the A-region is selected from the group consisting of SEQ ID NO: 20, SEQ ID NO: 21, and SEQ ID NO: 22.

29. A method according to claim 28, wherein the sequence (b1), (b2) or (b3) consists of five to eighteen amino acids present in AA₁ through AA₁₈ or a subsequence thereof comprising at least five amino acids, wherein:
AA₁ is selected from the group consisting of glycine and alanine;
AA₂ is selected from the group consisting of isoleucine, leucine, methionine and valine;
AA₃ is selected from the group consisting of isoleucine, leucine, methionine and valine;
AA₄ is selected from the group consisting of methionine, isoleucine, leucine and valine;
AA₅ is selected from the group consisting of lysine and arginine;
AA₆ is selected from the group consisting of aspartic acid, serine, glycine, glutamic acid, glutamine, asparagine, threonine, alanine and an aliphatic, substituted aliphatic, benzyl, substituted benzyl, aromatic or substituted aromatic ester of a glutamic or aspartic acid;
AA₇ is selected from the group consisting of phenylalanine, leucine, tryptophan, tyrosine, isoleucine, methionine and valine;
AA₈ is selected from the group consisting of serine, histidine, asparagine, threonine, glutamic acid, aspartic acid, glutamine and an aliphatic, substituted aliphatic, benzyl, substituted benzyl, aromatic or substituted aromatic ester of a glutamic or aspartic acid;
AA₉ is histidine;
AA₁₀ is proline;
AA₁₁ is selected from the group consisting of asparagine, glutamine, glutamic acid, aspartic acid, and an aliphatic, substituted aliphatic, benzyl, substituted benzyl, aromatic or substituted aromatic ester of a glutamic or aspartic acid;
AA₁₂ is selected from the group consisting of valine, isoleucine, leucine and methionine;
AA₁₃ is selected from the group consisting of leucine, isoleucine, methionine and valine;
AA₁₄ is selected from the group consisting of serine, alanine, threonine and glycine;
AA₁₅ is selected from the group consisting of leucine, isoleucine, methionine and valine;
AA₁₆ is selected from the group consisting of leucine, isoleucine, methionine and valine;
AA₁₇ is selected from the group consisting of glycine and alanine; and
AA₁₈ is selected from the group consisting of isoleucine, valine, leucine and methionine.

30. A method according to claim 1, wherein the kinase is an EGF receptor protein kinase selected from the group consisting of SRC, LYN, HCK and LCK protein and the sequence of the A-region is selected from the group consisting of SEQ ID NO: 23, SEQ ID NO: 24, SEQ ID NO: 25 and SEQ ID NO: 26.

31. A method according to claim 1, wherein the kinase is Ref-receptor protein kinase and the sequence of the A-region is represented by SEQ ID NO: 27.

32. A method according to claims 30 or 31, wherein the sequence (b1), (b2) or (b3) consists of five to eighteen amino acids present through AA₁ through AA₁₈, wherein:
AA₁ is selected from the group consisting of alanine, methionine, glycine, isoleucine, leucine and valine;
AA₂ is selected from the group consisting of tyrosine, leucine, phenylalanine, tryptophan, isoleucine, methionine and valine;
AA₃ is selected from the group consisting of valine, alanine, isoleucine, leucine, methionine and glycine;
AA₄ is selected from the group consisting of methionine, isoleucine, leucine and valine;
AA₅ is selected from the group consisting of alanine and glycine;
AA₆ is selected from the group consisting of serine, glycine, threonine and alanine;
AA₇ is selected from the group consisting of valine, leucine, methionine and isoleucine;
AA₈ is selected from the group consisting of aspartic acid, glycine, glutamic acid, glutamine, asparagine, alanine and an aliphatic, substituted aliphatic, benzyl, substituted benzyl, aromatic or substituted aromatic ester of a glutamic or aspartic acid;
AA₉ is selected from the group consisting of asparagine, serine, histidine, glutamic acid, aspartic acid, glutamine, threonine and an aliphatic, substituted aliphatic, benzyl, substituted benzyl, aromatic or substituted aromatic ester of a glutamic or aspartic acid;
AA₁₀ is selected from the group consisting of proline, alanine and glycine;
AA₁₁ is selected from the group consisting of histidine, tyrosine, phenylalanine and tryptophan;
AA₁₂ is selected from the group consisting of valine, isoleucine, leucine and methionine;
AA₁₃ is selected from the group consisting of cysteine, serine, valine, isoleucine, leucine and methionine;
AA₁₄ is selected from the group consisting of arginine and lysine;
AA₁₅ is selected from the group consisting of leucine, isoleucine, methionine and valine;
AA₁₆ is selected from the group consisting of leucine, isoleucine, methionine and valine;
AA₁₇ is selected from the group consisting of glycine and alanine; and
AA₁₈ is selected from the group consisting of isoleucine, leucine, valine, and methionine.

33. A method according to claim 1, wherein the kinase is an NGF receptor protein kinase selected from SRC, LYN, HCK or LCK protein and the sequence of the A-region is selected from the group consisting of SEQ ID NO: 28, SEQ ID NO: 29 and SEQ ID NO: 30.

34. A method according to claim 33, wherein the sequence (b1), (b2) or (b3) consists of five to eighteen amino acids present in in AA₁ through AA_{18,} wherein:
AA₁ is selected from the group consisting of valine, alanine, isoleucine, leucine, methionine and glycine;
AA₂ is selected from the group consisting of glutamic acid, aspartic acid, glutamine, asparagine and an aliphatic, substituted aliphatic, benzyl, substituted benzyl, aromatic or substituted aromatic ester of a glutamic or aspartic acid;
AA₃ is selected from the group consisting of leucine, isoleucine, methionine and valine;
AA₄ is selected from the group consisting of leucine, isoleucine, methionine and valine;
AA₅ is selected from the group consisting of threonine and serine;
AA₆ is selected from the group consisting of methionine, asparagine, isoleucine, leucine, valine, glutamic acid, aspartic acid, glutamine and an aliphatic, substituted aliphatic, benzyl, substituted benzyl, aromatic or substituted aromatic ester of a glutamic or aspartic acid;
AA₇ is selected from the group consisting of leucine, isoleucine, methionine and valine;
AA₈ is selected from the group consisting of glutamine, glutamic acid, aspartic acid, asparagine and an aliphatic, substituted aliphatic, benzyl, substituted benzyl, aromatic or substituted aromatic ester of a glutamic or aspartic acid;
AA₉ is histidine;
AA₁₀ is selected from the group consisting of glutamine, glutamic acid, aspartic acid, asparagine and an aliphatic, substituted aliphatic, benzyl, substituted benzyl, aromatic or substituted aromatic ester of a glutamic or aspartic acid;
AA₁₁ is histidine;
AA₁₂ is selected from the group consisting of isoleucine, leucine, methionine and valine;
AA₁₃ is selected from the group consisting of valine, isoleucine, leucine and methionine;
AA₁₄ is selected from the group consisting of arginine and lysine;
AA₁₅ is selected from the group consisting of phenylalanine, tryptophan and tyrosine;
AA₁₆ is selected from the group consisting of phenylalanine, tyrosine and tryptophan;
AA₁₇ is selected from the group consisting of glycine and alanine; and AA₁₈ is selected from the group consisting of valine, isoleucine, leucine and methionine.

35. The method according to claim 33, wherein the sequence (b1), (b2) or (b3) consists of five to eighteen amino acids present in AA₁ to AA₁₈ wherein:
AA₁ is selected from the group consisting of alanine and glycine;
AA₂ is selected from the group consisting of asparagine, glutamine, glutamic acid, and aspartic acid;
AA₃ is selected from the group consisting of valine, isoleucine, leucine and methionine;
AA₄ is selected from the group consisting of methionine, isoleucine, leucine and valine;
AA₅ is selected from the group consisting of glutamine, glutamic acid, aspartic acid, asparagine and an aliphatic, substituted aliphatic, benzyl, substituted benzyl, aromatic or substituted aromatic ester of a glutamic or aspartic acid;
AA₆ is selected from the group consisting of glutamine, glutamic acid, aspartic acid, asparagine and an aliphatic, substituted aliphatic, benzyl, substituted benzyl, aromatic or substituted aromatic ester of a glutamic or aspartic acid;
AA₇ is selected from the group consisting of leucine, isoleucine, methionine and valine;
AA₈ is selected from the group consisting of aspartic acid, glutamic acid, glutamine, asparagine and an aliphatic, substituted aliphatic, benzyl, substituted benzyl, aromatic or substituted aromatic ester of a glutamic or aspartic acid;
AA₉ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₀ is proline;
AA₁₁ is selected from the group consisting of tyrosine, phenylalanine and tryptophan;
AA₁₂ is selected from the group consisting of isoleucine, leucine, methionine and valine;
AA₁₃ is selected from the group consisting of valine, isoleucine, leucine and methionine;
AA₁₄ is selected from the group consisting of arginine and lysine;
AA₁₅ is selected from the group consisting of methionine, leucine, isoleucine and valine;
AA₁₆ is selected from the group consisting of isoleucine, leucine, methionine and valine;
AA₁₇ is selected from the group consisting of glycine and alanine; and
AA₁₈ is selected from the group consisting of isoleucine, valine, leucine and methionine.

36. The method according to claim 1, wherein the kinase is a Jak or Tyk protein kinase selected from the group consisting of SRC, LYN, HCK and LCK protein and the sequence of the A-region is selected from the group consisting of SEQ ID NO: 32, SEQ ID NO: 33, SEQ ID NO: 34 and SEQ ID NO: 35, respectively.

37. A method according to claim 3, wherein the kinase is IRK receptor protein kinase and wherein the A-region is a sequence represented by SEQ ID NO: 36.

38. A method according to claim 36 or 37, wherein the sequence (b1), (b2) or (b3) consists of five to eighteen amino acids present in AA₁ through AA₁₈ wherein:
AA₁ is selected from the group consisting of isoleucine, leucine, methionine and valine;
AA₂ is selected from the group consisting of glutamic acid, glutamine, aspartic acid, asparagine and an aliphatic, substituted aliphatic, benzyl, substituted benzyl, aromatic or substituted aromatic ester of a glutamic or aspartic acid;
AA₃ is selected from the group consisting of isoleucine, leucine, methionine and valine;
AA₄ is selected from the group consisting of leucine, isoleucine, methionine and valine;
AA₅ is selected from the group consisting of arginine and lysine;
AA₆ is selected from the group consisting of asparagine, serine, alanine, threonine, glutamine, glutamic acid, aspartic acid, glycine and an aliphatic, substituted aliphatic, benzyl, substituted benzyl, aromatic or substituted aromatic ester of a glutamic or aspartic acid;
AA₇ is selected from the group consisting of leucine, isoleucine, methionine and valine;
AA₈ is selected from the group consisting of tyrosine, glutamine, histidine, phenylalanine, tryptophan, glutamic acid, aspartic acid, asparagine and an aliphatic, substituted aliphatic, benzyl, substituted benzyl, aromatic or substituted aromatic ester of a glutamic or aspartic acid;
AA₉ is selected from the group consisting of histidine, serine and threonine; AA₁₀ is selected from the group consisting of glutamic acid, aspartic acid, asparagine, glutamine and an aliphatic, substituted aliphatic, benzyl, substituted benzyl, aromatic or substituted aromatic ester of a glutamic or aspartic acid;
AA₁₁ is selected from the group consisting of asparagine, phenylalanine, histidine, glutamic acid, aspartic acid, glutamine, tryptophan, tyrosine and an aliphatic, substituted aliphatic, benzyl, substituted benzyl, aromatic or substituted aromatic ester of a glutamic or aspartic acid;
AA₁₂ is selected from the group consisting of isoleucine, leucine, methionine and valine;
AA₁₃ is selected from the group consisting of valine, isoleucine, leucine and methionine;
AA₁₄ is selected from the group consisting of lysine and arginine;
AA₁₅ is selected from the group consisting of tyrosine, phenylalanine, and tryptophan;
AA₁₆ is selected from the group consisting of lysine and arginine;
AA₁₇ is selected from the group consisting of glycine and alanine; and
AA₁₈ is selected from the group consisting of isoleucine, valine, cysteine, leucine, methionine and serine.

39. A method according to claim 1, wherein the kinase is an activin receptor-like protein kinase selected from the group consisting of SRC, LYN, HCK and LCK protein and the sequence of the A-region is selected from the group consisting of SEQ ID NO: 37, SEQ ID NO: 38, SEQ ID NO: 39 and SEQ ID NO: 40.

40. A method according to claim 39, wherein the sequence (b1), (b2) or (b3) consists of five to eighteen amino acids present in AA₁ to AA₁₈ wherein:
AA₁ is selected from the group consisting of isoleucine, leucine, methionine and valine;
AA₂ is selected from the group consisting of tyrosine, phenylalanine and tryptophan;
AA₃ is selected from the group consisting of asparagine, glutamine, glutamic acid, aspartic acid and an aliphatic, substituted aliphatic, benzyl, substituted benzyl, aromatic or substituted aromatic ester of a glutamic or aspartic acid;
AA₄ is selected from the group consisting of threonine and serine;
AA₅ is selected from the group consisting of valine, isoleucine, leucine and methionine;
AA₆ is selected from the group consisting of leucine, methionine, isoleucine, and valine;
AA₇ is selected from the group consisting of leucine, methionine, isoleucine and valine;
AA₈ is selected from the group consisting of arginine and lysine;
AA₉ is histidine;
AA₁₀ is selected from the group consisting of aspartic acid, glutamic acid, asparagine, glutamine and an aliphatic, substituted aliphatic, benzyl, substituted benzyl, aromatic or substituted aromatic ester of a glutamic or aspartic acid;
AA₁₁ is selected from the group consisting of asparagine, glutamine, glutamic acid, aspartic acid and an aliphatic, substituted aliphatic, benzyl, substituted benzyl, aromatic or substituted aromatic ester of a glutamic or aspartic acid;
AA₁₂ is selected from the group consisting of isoleucine, leucine, methionine and valine;
AA₁₃ is selected from the group consisting of leucine, isoleucine, methionine and valine;
AA₁₄ is selected from the group consisting of glycine and alanine;
AA₁₅ is selected from the group consisting of phenylalanine, tryptophan and tyrosine;
AA₁₆ is selected from the group consisting of isoleucine, leucine, methionine and valine;
AA₁₇ is selected from the group consisting of alanine and glycine; and
AA₁₈ is selected from the group consisting of serine, alanine, glycine and threonine.

41. A method according to claim 1, wherein the kinase is a discoidin domain receptor protein selected from the group consisting of SRC, LYN, HCK and LCK protein and the sequence of the A-region is selected from the group consisting of SEQ ID NO: 41 and SEQ ID NO: 42.

42. A method according to claim 41, wherein the sequence (b1), (b2) or (b3) consists of five to eighteen amino acids present in AA₁ to AA₁₈ wherein:
AA₁ is selected from the group consisting of valine, isoleucine, leucine and methionine;
AA₂ is selected from the group consisting of lysine and arginine;
AA₃ is selected from the group consisting of isoleucine, leucine, methionine and valine;
AA₄ is selected from the group consisting of methionine, isoleucine, leucine and valine;
AA₅ is selected from the group consisting of serine and threonine;
AA₆ is selected from the group consisting of arginine and lysine;
AA₇ is selected from the group consisting of leucine, isoleucine, methionine and valine;
AA₈ is selected from the group consisting of lysine and arginine;
AA₉ is selected from the group consisting of aspartic acid, glutamic acid, asparagine, glutamine and an aliphatic, substituted aliphatic, benzyl, substituted benzyl, aromatic or substituted aromatic ester of a glutamic or aspartic acid;
AA₁₀ is proline;
AA₁₁ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₂ is selected from the group consisting of isoleucine, leucine, methionine and valine;
AA₁₃ is selected from the group consisting of isoleucine, leucine, methionine and valine;
AA₁₄ is selected from the group consisting of arginine, histidine and lysine;
AA₁₅ is selected from the group consisting of leucine, isoleucine, methionine and valine;
AA₁₆ is selected from the group consisting of leucine, isoleucine, methionine and valine;
AA₁₇ is selected from the group consisting of glycine, serine, alanine and threonine; and
AA₁₈ is selected from the group consisting of valine, isoleucine, leucine and methionine.

43. A method of detecting a ligand that binds to the A-region of a protein kinase comprising:
(a) providing a compound according to claim 7 or 8;
(b) incubating said compound with a sample, to be tested for the presence of said ligand, for a time sufficient for said ligand to bind to said compound; and
(c) detecting any said ligand-said compound binding pair that has been formed in step (b), wherein the presence of said ligand-said compound derivative binding pair establishes the existence of said ligand in said sample.

44. The method of claim 43, further comprising the following steps after step (c):
(d) separating said ligand from said compound; and
(e)determining the structure of said ligand, thereby identifying said ligand.

45. A pharmaceutical composition, comprising a pharmaceutically acceptable carrier and, as an active ingredient, at least one of the compounds of claim 7 or 8.

46. The pharmaceutical composition according to claim 45 which is for the treatment of a disease, wherein a therapeutically beneficial effect may be evident by the modulation of a signal transduction associated with a kinase, wherein the kinase from which the A-region is determined, is the kinase associated with said signal transduction

47. The pharmaceutical composition according to claim 47, wherein the disease is selected the group consisting of diabetes, cancer, obesity, restenosis, tissue remodeling including: improved bone healing, prevention of alopecia, reduced scarring, osteoporosis, neurodegenerative disease, autoimmune disease, inflammation, restenosis an, atherosclerosis, skin disorders, diseases of the central nervous system, inflammatory disorders, autoimmune diseases and other immune disorders, osteoporosis and cardiovascular diseases.

48. The pharmaceutical composition according to claim 47, wherein the disease is diabetes and the kinase is IRK.


SEQUENCE LISTING (133 sequences)

SEQ ID NO: 1 - protein, 18 aa, Artificial Sequence
  c-Src
AQVMKKLRHEKLVQLYAV

SEQ ID NO: 2 - protein, 18 aa, Artificial Sequence
  c-Yes
AQIMKKLRHDKLVPLYAV

SEQ ID NO: 3 - protein, 18 aa, Artificial Sequence
  Fyn
AQIMKKLKHDKLVQLYAV

SEQ ID NO: 4 - protein, 18 aa, Artificial Sequence
  c-Fgr
AQVMKLLRHDKLVQLYAV

SEQ ID NO: 5 - protein, 18 aa, Artificial Sequence
  Lyn
ANLMKTLQHDKLVRLYAV

SEQ ID NO: 6 - protein, 18 aa, Artificial Sequence
  Hck
ANVMKTLQHDKLVKLHAV

SEQ ID NO: 7 - protein, 18 aa, Artificial Sequence
  Lck
ANLMKQLQHQRLVRLYAV

SEQ ID NO: 8 - protein, 18 aa, Artificial Sequence
  Csk
ASVMTQLRHSNLVQLLGV

SEQ ID NO: 9 - protein, 18 aa, Artificial Sequence
  Matk
TAVMTKMQHENLVRLLGV

SEQ ID NO: 10 - protein, 18 aa, Artificial Sequence
  Fak
ALTMRQFDHPHIVKLIGV

SEQ ID NO: 11 - protein, 18 aa, Artificial Sequence
  c-Abl
AAVMKEIKHPNLVQLLGV

SEQ ID NO: 12 - protein, 19 aa, Artificial Sequence
  Tie/Tek
LEVLCKLGHHPNIINLLGA

SEQ ID NO: 13 - protein, 19 aa, Artificial Sequence
  FGFR
MEMMKMIGKHKNIINLLGA

SEQ ID NO: 14 - protein, 19 aa, Artificial Sequence
  FGFR
MEVMKLIGRHKNIINLLGV

SEQ ID NO: 15 - protein, 19 aa, Artificial Sequence
  PDGFR-a
LKIMTHLGPHLNIVNLLGA

SEQ ID NO: 16 - protein, 19 aa, Artificial Sequence
  PDGFR-b
LKIMSHLGPHLNVVNLLGA

SEQ ID NO: 17 - protein, 19 aa, Artificial Sequence
  Flt1
LKILTHIGHHLNVVNLLGA

SEQ ID NO: 18 - protein, 19 aa, Artificial Sequence
  Flt4
LKILIHIGNHLNVVNLLGA

SEQ ID NO: 19 - protein, 19 aa, Artificial Sequence
  Flk1
LKILIHIGHHLNVVNLLGA

SEQ ID NO: 20 - protein, 18 aa, Artificial Sequence
  c-Met
GIIMKDFSHPNVLSLLGI

SEQ ID NO: 21 - protein, 18 aa, Artificial Sequence
  c-Sea
GILMKSFHHPQVLSLLGV

SEQ ID NO: 22 - protein, 18 aa, Artificial Sequence
  Ron
GLLMRGLNHPNVLALIGI

SEQ ID NO: 23 - protein, 18 aa, Artificial Sequence
  EGFR
AYVMASVDNPHVCRLLGI

SEQ ID NO: 24 - protein, 18 aa, Artificial Sequence
  ErbB2
AYVMAGVGSPYVSRLLGI

SEQ ID NO: 25 - protein, 18 aa, Artificial Sequence
  ErbB3
MLAIGSLDHAHIVRLLGL

SEQ ID NO: 26 - protein, 18 aa, Artificial Sequence
  ErbB4
ALIMASMDHPHLVRLLGV

SEQ ID NO: 27 - protein, 18 aa, Artificial Sequence
  Ret
FNVLKQVNHPHVIKLYGA

SEQ ID NO: 28 - protein, 18 aa, Artificial Sequence
  Trk-NGFR
VELLTMLQHQHIVRFFGV

SEQ ID NO: 29 - protein, 18 aa, Artificial Sequence
  TrkB/TrkC
AELLTNLQHEHIVKFYGV

SEQ ID NO: 30 - protein, 18 aa, Artificial Sequence
  Syk
ANVMQQLDNPYIVRMIGI

SEQ ID NO: 31 - protein, 18 aa, Artificial Sequence
  Zap70
AQIMEQLDNPYIVRLIGV

SEQ ID NO: 32 - protein, 18 aa, Artificial Sequence
  Jak1
IEILRNLYHENIVKYKGI

SEQ ID NO: 33 - protein, 18 aa, Artificial Sequence
  Jak2
IEILKSLQHDNIVKYKGV

SEQ ID NO: 34 - protein, 18 aa, Artificial Sequence
  Jak3
IQILKALHSDFIVKYRGV

SEQ ID NO: 35 - protein, 18 aa, Artificial Sequence
  Tyk2
IDILRTLYHEHIIKYKGC

SEQ ID NO: 36 - protein, 18 aa, Artificial Sequence
  IRK
ASVMKGFTCHHVVRLLGV

SEQ ID NO: 37 - protein, 18 aa, Artificial Sequence
  ALK1
IYNTVLLRHDNILGFIAS

SEQ ID NO: 38 - protein, 18 aa, Artificial Sequence
  ALK2
LYNTVMLRHENILGFIAS

SEQ ID NO: 39 - protein, 18 aa, Artificial Sequence
  ALK3/ALK6
IYQTVLMRHENILGFIAA

SEQ ID NO: 40 - protein, 18 aa, Artificial Sequence
  ALK4/ALK5
IYQTVMLRHENILGFIAA

SEQ ID NO: 41 - protein, 18 aa, Artificial Sequence
  DDR1
VKIMSRLKDPNIIRLLGV

SEQ ID NO: 42 - protein, 18 aa, Artificial Sequence
  DDR2
IKIMSRLKDPNIIHLLSV

SEQ ID NO: 43 - protein, 18 aa, Artificial Sequence
  ACK
VNAMHSLDHRNLIRLYGV

SEQ ID NO: 44 - protein, 18 aa, Artificial Sequence
  Eph-B4
ASIMGQFEHPNIIRLEGV

SEQ ID NO: 45 - protein, 18 aa, Artificial Sequence
  ITK/TSK
AEVMMKLSHPKLVQLYGV

SEQ ID NO: 46 - protein, 18 aa, Artificial Sequence
  Plk
ISIHRSLAHQHVVGFHGF

SEQ ID NO: 47 - protein, 18 aa, Artificial Sequence
  Plx1
IEILATCNHHFIVKLLGA

SEQ ID NO: 48 - protein, 18 aa, Artificial Sequence
  Polo
ITIHRSLNHPNIVKFHNY

SEQ ID NO: 49 - protein, 18 aa, Artificial Sequence
  SNK
IELHRILHHKHVVQFYHY

SEQ ID NO: 50 - protein, 18 aa, Artificial Sequence
  CDC5
IQIHKSMSHPNIVQFIDC

SEQ ID NO: 51 - protein, 18 aa, Artificial Sequence
  Sak
VKIHCQLKHPSVLELYNY

SEQ ID NO: 52 - protein, 18 aa, Artificial Sequence
  Prk/Fnk
IELHRDLQHRHIVRFSHH

SEQ ID NO: 53 - protein, 18 aa, Artificial Sequence
  Plol
IKVHQSMSHPNIVGFIDC

SEQ ID NO: 54 - protein, 11 aa, Artificial Sequence
  plk
GSLAHQHVVGF

SEQ ID NO: 55 - protein, 11 aa, Artificial Sequence
  plx1
GTCNHHFIVKL

SEQ ID NO: 56 - protein, 11 aa, Artificial Sequence
  polo
GSLNHPNIVKF

SEQ ID NO: 57 - protein, 11 aa, Artificial Sequence
  snk
GILHHKHVVQF

SEQ ID NO: 58 - protein, 11 aa, Artificial Sequence
  CDC5
GSMSHPNIVQF

SEQ ID NO: 59 - protein, 11 aa, Artificial Sequence
  Sak
GQLKHPSVLEL

SEQ ID NO: 60 - protein, 11 aa, Artificial Sequence
  prk
GDLQHRHIVRF

SEQ ID NO: 61 - protein, 11 aa, Artificial Sequence
  plol
GSMSHPNIVGF

SEQ ID NO: 62 - protein, 11 aa, Artificial Sequence
  Alk1
GLLRHDNILGF

SEQ ID NO: 63 - protein, 11 aa, Artificial Sequence
  c-Src
GKLRHEKLVQL

SEQ ID NO: 64 - protein, 11 aa, Artificial Sequence
  c-Yes
GKLRHDKLVPL

SEQ ID NO: 65 - protein, 11 aa, Artificial Sequence
  Fyn
GKLKHDKLVQL

SEQ ID NO: 66 - protein, 11 aa, Artificial Sequence
  c-Fgr
GLLRHDKLVQL

SEQ ID NO: 67 - protein, 11 aa, Artificial Sequence
  Lyn
GTLQHDKLVRL

SEQ ID NO: 68 - protein, 11 aa, Artificial Sequence
  Hck
GTLQHDKLVKL

SEQ ID NO: 69 - protein, 11 aa, Artificial Sequence
  Lck
GQLQHQRLVRL

SEQ ID NO: 70 - protein, 11 aa, Artificial Sequence
  Csk
GQLRHSNLVQL

SEQ ID NO: 71 - protein, 11 aa, Artificial Sequence
  Matk
GKMQHENLVRL

SEQ ID NO: 72 - protein, 11 aa, Artificial Sequence
  Fak
GQFDHPHIVKL

SEQ ID NO: 73 - protein, 11 aa, Artificial Sequence
  c-Abl
GEIKHPNLVQL

SEQ ID NO: 74 - protein, 12 aa, Artificial Sequence
  Tie
GKLGHNPNIINL

SEQ ID NO: 75 - protein, 12 aa, Artificial Sequence
  PDGFR-b
GHLGPHLNVVNL

SEQ ID NO: 76 - protein, 12 aa, Artificial Sequence
  PDGFR-a
GHLGPHLNIVNL

SEQ ID NO: 77 - protein, 12 aa, Artificial Sequence
  Flt1
GHIGHHLNVVNL

SEQ ID NO: 78 - protein, 12 aa, Artificial Sequence
  Flt4
GHIGNHLNVVNL

SEQ ID NO: 79 - protein, 12 aa, Artificial Sequence
  Flg
GMIGKHKNIINL

SEQ ID NO: 80 - protein, 12 aa, Artificial Sequence
  FGFR-4
GLIGRHKNIINL

SEQ ID NO: 81 - protein, 11 aa, Artificial Sequence
  c-MET
GDFSHPNVLSL

SEQ ID NO: 82 - protein, 11 aa, Artificial Sequence
  c-SEA
GSFHHPQVLSL

SEQ ID NO: 83 - protein, 11 aa, Artificial Sequence
  Ron
GGLNHPNVLAL

SEQ ID NO: 84 - protein, 11 aa, Artificial Sequence
  EGFR
GSVDNPHVCRL

SEQ ID NO: 85 - protein, 11 aa, Artificial Sequence
  ErbB2
GGVGSPYVSRL

SEQ ID NO: 86 - protein, 11 aa, Artificial Sequence
  ErbB3
GSLDHAHIVRL

SEQ ID NO: 87 - protein, 11 aa, Artificial Sequence
  ErbB4
GSMDHPHLVRL

SEQ ID NO: 88 - protein, 11 aa, Artificial Sequence
  Ret
GQVNHPHVIKL

SEQ ID NO: 89 - protein, 11 aa, Artificial Sequence
  Trk-NGFR
GMLQHQHIVRF

SEQ ID NO: 90 - protein, 11 aa, Artificial Sequence
  Trk-NGFR
GNLQHEHIVKF

SEQ ID NO: 91 - protein, 11 aa, Artificial Sequence
  Trk-NGFR
GDLQHRHIVRF

SEQ ID NO: 92 - protein, 11 aa, Artificial Sequence
  Trk-NGFR
GNLQHRHIVRF

SEQ ID NO: 93 - protein, 11 aa, Artificial Sequence
  Syk
GQLDNPYIVRM

SEQ ID NO: 94 - protein, 11 aa, Artificial Sequence
  Zap70
GQLDNPYIVRL

SEQ ID NO: 95 - protein, 11 aa, Artificial Sequence
  Jak1
GNLYHENIVKY

SEQ ID NO: 96 - protein, 11 aa, Artificial Sequence
  Jak2
GSLQHDNIVKY

SEQ ID NO: 97 - protein, 11 aa, Artificial Sequence
  Jak3
GALHSDFIVKY

SEQ ID NO: 98 - protein, 10 aa, Artificial Sequence
  IRK
GFTCHHVVRL

SEQ ID NO: 99 - protein, 10 aa, Artificial Sequence
  IRK
GFTSHHVVRL

SEQ ID NO: 100 - protein, 12 aa, Artificial Sequence
  IRK
GGFTCHHVVRLL

SEQ ID NO: 101 - protein, 12 aa, Artificial Sequence
  Irk
GFTCHHVVRRLL

SEQ ID NO: 102 - protein, 11 aa, Artificial Sequence
  Irk
GGFTCHHVVRL

SEQ ID NO: 103 - protein, 10 aa, Artificial Sequence
  Irk
GGFTCHHVVR

SEQ ID NO: 104 - protein, 11 aa, Artificial Sequence
  Irk
GFTCHHVVRLL

SEQ ID NO: 105 - protein, 9 aa, Artificial Sequence
  Irk
GFTCHHVVR

SEQ ID NO: 106 - protein, 8 aa, Artificial Sequence
  Irk
GFTCHHVV

SEQ ID NO: 107 - protein, 12 aa, Artificial Sequence
  Irk
GGGFTCHHVVRL

SEQ ID NO: 108 - protein, 11 aa, Artificial Sequence
  Irk Position 11 is benzoylated
GGFTCHHVVRK

SEQ ID NO: 109 - protein, 11 aa, Artificial Sequence
  Irk
GGFTSHHVVRL

SEQ ID NO: 110 - protein, 9 aa, Artificial Sequence
  Irk
GGFTCHHVV

SEQ ID NO: 111 - protein, 11 aa, Artificial Sequence
  Irk Position 5 is alanine-beta-amino-cysteine
GGFTCHHVVRL

SEQ ID NO: 112 - protein, 11 aa, Artificial Sequence
  Irk Position 5 is lysine-epsilon-amino-cysteine
GGFTCHHVVRL

SEQ ID NO: 113 - protein, 10 aa, Artificial Sequence
  Irk
GGFTHHVVRL

SEQ ID NO: 114 - protein, 11 aa, Artificial Sequence
  Alk2
GMLRHENILGF

SEQ ID NO: 115 - protein, 11 aa, Artificial Sequence
  Alk3
GLMRHENILGF

SEQ ID NO: 116 - protein, 11 aa, Artificial Sequence
  TrkB
GNLQHEHIVKF

SEQ ID NO: 117 - protein, 11 aa, Artificial Sequence
  DDR1
GRLKDPNIIRL

SEQ ID NO: 118 - protein, 11 aa, Artificial Sequence
  DDR2
GRLKDPNIIHL

SEQ ID NO: 119 - protein, 11 aa, Artificial Sequence
  Tyk2
GTLYHEHIIKY

SEQ ID NO: 120 - protein, 11 aa, Artificial Sequence
  Eph-B4
GQFEHPNIIRL

SEQ ID NO: 121 - protein, 11 aa, Artificial Sequence
  ITK/TSK
GKLSHPKLVQL

SEQ ID NO: 122 - protein, 11 aa, Artificial Sequence
  ACK
GSLDHRNLIRL

SEQ ID NO: 123 - protein, 11 aa, Artificial Sequence
  IRK
GGFTCHHVVRL

SEQ ID NO: 124 - protein, 12 aa, Artificial Sequence
  IRK
GGAFTCHHVVRL

SEQ ID NO: 125 - protein, 12 aa, Artificial Sequence
  IRK
GGGATCHHVVRL

SEQ ID NO: 126 - protein, 12 aa, Artificial Sequence
  IRK
GGGFACHHVVRL

SEQ ID NO: 127 - protein, 12 aa, Artificial Sequence
  IRK
GGGFTAHHVVRL

SEQ ID NO: 128 - protein, 12 aa, Artificial Sequence
  IRK
GGGFTCAHVVRL

SEQ ID NO: 129 - protein, 12 aa, Artificial Sequence
  IRK
GGGFTCHAVVRL

SEQ ID NO: 130 - protein, 12 aa, Artificial Sequence
  IRK
GGGFTCHHAVRL

SEQ ID NO: 131 - protein, 12 aa, Artificial Sequence
  IRK
GGGFTCHHVARL

SEQ ID NO: 132 - protein, 12 aa, Artificial Sequence
  IRK
GGGFTCHHVVAL

SEQ ID NO: 133 - protein, 12 aa, Artificial Sequence
  IRK
GGGFTCHHVVRA